SEQUENCE LISTING (75 sequences)

SEQ ID NO: 1 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 2 - protein, 247 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTERESLGRHRWLTRPRTRTTRRDGAS

SEQ ID NO: 3 - protein, 723 aa, Homo sapiens; identical to SEQ ID NO: 1

SEQ ID NO: 4 - DNA, 2389 bp, Homo sapiens
agatataaggcttggaagccagcagctgcgactcccgagacccccccaccagaaggccatggtctccccacggatgtccgggctcctctcccagactgtgatcctagcgctcattttcctcccccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggtagcgacgccccttttccccccgctacacactgggcgcgctgggcagaggcagcacctgctttttccctacccttcctcgattctgtccgtgaaatgaattgggtagagtctctggaaggttttaagcccattttcagttctaacttactttcatcctattttgcatccctcttatcgttttgagctacctgccatcttctctttgaaaaacctatgggcttgaggaggtcacgatgccgactccgccagagcttttccactgattgtactcagcggggaggcaggggaggcagaggggcagcctctctaatgcttcctactcattttgtttctaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatcagttttgactaacaaaaaa

SEQ ID NO: 5 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 6 - DNA, 2052 bp, Homo sapiens
agatataaggcttggaagccagcagctgcgactcccgagacccccccaccagaaggccatggtctccccacggatgtccgggctcctctcccagactgtgatcctagcgctcattttcctcccccagacacggcccgctggcgtcttcgagctgcagatccactctttcgggccgggtccaggccctggggccccgcggtccccctgcagcgcccggctcccctgccgcctcttcttcagagtctgcctgaagcctgggctctcagaggaggccgccgagtccccgtgcgccctgggcgcggcgctgagtgcgcgcggaccggtctacaccgagcagcccggagcgcccgcgcctgatctcccactgcccgacggcctcttgcaggtgcccttccgggacgcctggcctggcaccttctctttcatcatcgaaacctggagagaggagttaggagaccagattggagggcccgcctggagcctgctggcgcgcgtggctggcaggcggcgcttggcagccggaggcccgtgggcccgggacattcagcgcgcaggcgcctgggagctgcgcttctcgtaccgcgcgcgctgcgagccgcctgccgtcgggaccgcgtgcacgcgcctctgccgtccgcgcagcgccccctcgcggtgcggtccgggactgcgcccctgcgcaccgctcgaggacgaatgtgaggcgccgctggtgtgccgagcaggctgcagccctgagcatggcttctgtgaacagcccggtgaatgccgatgcctagagggctggactggacccctctgcacggtccctgtctccaccagcagctgcctcagccccaggggcccgtcctctgctaccaccggatgccttgtccctgggcctgggccctgtgacgggaacccgtgtgccaatggaggcagctgtagtgagacacccaggtcctttgaatgcacctgcccgcgtgggttctacgggctgcggtgtgaggtgagcggggtgacatgtgcagatggaccctgcttcaacggcggcttgtgtgtcgggggtgcagaccctgactctgcctacatctgccactgcccacccggtttccaaggctccaactgtgagaagagggtggaccggtgcagcctgcagccatgccgcaatggcggactctgcctggacctgggccacgccctgcgctgccgctgccgcgccggcttcgcgggtcctcgctgcgagcacgacctggacgactgcgcgggccgcgcctgcgctaacggcggcacgtgtgtggagggcggcggcgcgcaccgctgctcctgcgcgctgggcttcggcggccgcgactgccgcgagcgcgcggacccgtgcgccgcgcgcccctgtgctcacggcggccgctgctacgcccacttctccggcctcgtctgcgcttgcgctcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattgtgatcagttttgactaacaaaaaa

SEQ ID NO: 7 - protein, 587 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREA

SEQ ID NO: 8 - DNA, 3420 bp, Homo sapiens
aggtttcagtagcggcgctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagatgccactgggcactgccctgccagtagtggccttcagggggctccttccggggctccggcctgttttccagagagagtggcagtagccccatggggcccggagctgctgtggcctccactggcatccgtgtttccaaaagtgcctttggcccaggctccacggcgacagttgggcccaaatcagaaaggagagagggggccaatgagggcagggcctcctgtgggctggaaaaccactgggtgcgtctcttgctggggtttgccctggaggtgaggtgagtgctcgagggaggggagtgctttctgccccatgcctccaactactgtatgcaggcctggctctctggtctaggccctttgggcaagaatgtccgtctacccggcttccaccaccctctggccctgggcttctgtaagcagacaggcagagggcctgcccctcccaccagccaagggtgccaggcctaactggggcactcagggcagtgtgttggaaattccactgagggggaaatcaggtgctgcggccgcctgggccctttcctccctcaagcccatctccacaacctcgagcctgggctctggtccactactgccccagaccaccctcaaagctggtcttcagaaatcaataatatgagtttttattttgtttttttttttttttttgtagtttattttggagtctagtatttcaataatttaagaatcagaagcactgacctttctacattttataacattattttgtatataatgtgtatttataatatgaaaca

SEQ ID NO: 9 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 10 - protein, 383 aa, Homo sapiens
MTATEALLRVLLLLLAFGHSTYGAECFPACNPQNGFCEDDNVCRCQPGWQGPLCDQCVTSPGCLHGLCGEPGQCICTDGWDGELCDRDVRACSSAPCANNGTCVSLDDGLYECSCAPGYSGKDCQKKDGPCVINGSPCQHGGTCVDDEGRASHASCLCPPGFSGNFCEIVANSCTPNPCENDGVCTDIGGDFRCRCPAGFIDKTCSRPVTNCASSPCQNGGTCLQHTQVSYECLCKPEFTGLTCVKKRALSPQQVTRLPSGYGLAYRLTPGVHELPVQQPEHRILKVSMKELNKKTPLLTEGQAICFTILGVLTSLVVLGTVGIVFLNKCETWVSNLRYNHMLRKKKNLLLQYNSGEDLAVNIIFPEKIDMTTFSKEAGDEEI

SEQ ID NO: 11 - DNA, 1599 bp, Homo sapiens
gaaaagggcggcgcgcgcggcggcggcggcagctccccggcagcggcggtggagagcgcagcgcgcagcccggtgcagccctggctttcccctcgctgcgcgcccgcgccccctttcgcgtccgcaaccagaagcccagtgcggcgccaggagccggacccgcgcccgcaccgctcccgggaccgcgaccccggccgcccagagatgaccgcgaccgaagccctcctgcgcgtcctcttgctcctgctggctttcggccacagcacctatggggctgaatgcttcccggcctgcaacccccaaaatggattctgcgaggatgacaatgtttgcaggtgccagcctggctggcagggtcccctttgtgaccagtgcgtgacctctcccggctgccttcacggactctgtggagaacccgggcagtgcatttgcaccgacggctgggacggggagctctgtgatagagatgttcgggcctgctcctcggccccctgtgccaacaacgggacctgcgtgagcctggacgatggcctctatgaatgctcctgtgcccccgggtactcgggaaaggactgccagaaaaaggacgggccctgtgtgatcaacggctccccctgccagcacggaggcacctgcgtggatgatgagggccgggcctcccatgcctcctgcctgtgcccccctggcttctcaggcaatttctgcgagatcgtggccaacagctgcacccccaacccatgcgagaacgacggcgtctgcactgacattgggggcgacttccgctgccggtgcccagccggcttcatcgacaagacctgcagccgcccggtgaccaactgcgccagcagcccgtgccagaacgggggcacctgcctgcagcacacccaggtgagctacgagtgtctgtgcaagcccgagttcacaggtctcacctgtgtcaagaagcgcgcgctgagcccccagcaggtcacccgtctgcccagcggctatgggctggcctaccgcctgacccctggggtgcacgagctgccggtgcagcagccggagcaccgcatcctgaaggtgtccatgaaagagctcaacaagaaaacccctctcctcaccgagggccaggccatctgcttcaccatcctgggcgtgctcaccagcctggtggtgctgggcactgtgggtatcgtcttcctcaacaagtgcgagacctgggtgtccaacctgcgctacaaccacatgctgcggaagaagaagaacctgctgcttcagtacaacagcggggaggacctggccgtcaacatcatcttccccgagaagatcgacatgaccaccttcagcaaggaggccggcgacgaggagatctaagcagcgttcccacagccccctctagattcttggagttccgcagagcttactatacgcggtctgtcctaatctttgtggtgttcgctatctcttgtgtcaaatctggtgaacgctacgcttacatatattgtctttgtgctgctgtgtgacaaacgcaatgcaaaaacaatcctctttctctctcttaatgcatgatacagaataataataagaatttcatctttaaatgagtaaaaaaaaaaa

SEQ ID NO: 12 - protein, 383 aa, Homo sapiens
MPSGCRCLHLVCLLCILGAPGQPVRADDCSSHCDLAHGCCAPDGSCRCDPGWEGLHCERCVRMPGCQHGTCHQPWQCICHSGWAGKFCDKDEHICTTQSPCQNGGQCMYDGGGEYHCVCLPGFHGRDCERKAGPCEQAGSPCRNGGQCQDDQGFALNFTCRCLVGFVGARCEVNVDDCLMRPCANGATCLDGINRFSCLCPEGFAGRFCTINLDDCASRPCQRGARCRDRVHDFDCLCPSGYGGKTCELVLPVPDPPTTVDTPLGPTSAVVVPATGPAPHSAGAGLLRISVKEVVRRQEAGLGEPSLVALVVFGALTAALVLATVLLTLRAWRRGVCPPGPCCYPAPHYAPACQDQECQVSMLPAGLPLPRDLPPEPGKTTAL

SEQ ID NO: 13 - protein, 383 aa, Homo sapiens; identical to SEQ ID NO: 12

SEQ ID NO: 14 - DNA, 1574 bp, Homo sapiens
agattcccgagcgctcggctcgcatggcagccgcttcggcgcccggccccgcggccagctaggggcggccccgcgctccctcacggcccctcggcggcgcccgtcggatccggcctctctctgcgccccggggcgcgccacctccccgccggaggtgtccacgcgtccggccgtccatccgtccgtccctcctggggccggcgctgaccatgcccagcggctgccgctgcctgcatctcgtgtgcctgttgtgcattctgggggctcccggtcagcctgtccgagccgatgactgcagctcccactgtgacctggcccacggctgctgtgcacctgacggctcctgcaggtgtgacccgggctgggaggggctgcactgtgagcgctgtgtgaggatgcctggctgccagcacggtacctgccaccagccatggcagtgcatctgccacagtggctgggcaggcaagttctgtgacaaagatgaacatatctgtaccacgcagtccccctgccagaatggaggccagtgcatgtatgacgggggcggtgagtaccattgtgtgtgcttaccaggcttccatgggcgtgactgcgagcgcaaggctggaccctgtgaacaggcaggctccccatgccgcaatggcgggcagtgccaggacgaccagggctttgctctcaacttcacgtgccgctgcttggtgggctttgtgggtgcccgctgtgaggtaaatgtggatgactgcctgatgcggccttgtgctaacggtgccacctgccttgacggcataaaccgcttctcctgcctctgtcctgagggctttgctggacgcttctgcaccatcaacctggatgactgtgccagccgcccatgccagagaggggcccgctgtcgggaccgtgtccacgacttcgactgcctctgccccagtggctatggtggcaagacctgtgagcttgtcttacctgtcccagaccccccaaccacagtggacacccctctagggcccacctcagctgtagtggtacctgccacggggccagccccccacagcgcaggggctggtctgctgcggatctcagtgaaggaggtggtgcggaggcaagaggctgggctaggtgagcctagcttggtggccctggtggtgtttggggccctcactgctgccctggttctggctactgtgttgctgaccctgagggcctggcgccggggtgtctgcccccctggaccctgttgctaccctgccccacactatgctccagcgtgccaggaccaggagtgtcaggttagcatgctgccagcagggctccccctgccacgtgacttgccccctgagcctggaaagaccacagcactgtgatggaggtgggggctttctggcccccttcctcacctcttccacccctcagactggagtggtccgttctcaccacccttcagcttgggtacacacacagaggagacctcagcctcacaccagaaatattatttttttaatacacagaatgtaagatggaattttatcaaataaaactatgaaaatgcaaaaaaaaaaaaaaaaaaaaaaaaaaaa

SEQ ID NO: 15 - DNA, 1470 bp, Homo sapiens
ggcaacgtggacaggaagaagcggagggcgaggaggagcagaggagcacacagatgaagcaggtgtccacgcgtccggccgtccatccgtccgtccctcctggggccggcgctgaccatgcccagcggctgccgctgcctgcatctcgtgtgcctgttgtgcattctgggggctcccggtcagcctgtccgagccgatgactgcagctcccactgtgacctggcccacggctgctgtgcacctgacggctcctgcaggtgtgacccgggctgggaggggctgcactgtgagcgctgtgtgaggatgcctggctgccagcacggtacctgccaccagccatggcagtgcatctgccacagtggctgggcaggcaagttctgtgacaaagatgaacatatctgtaccacgcagtccccctgccagaatggaggccagtgcatgtatgacgggggcggtgagtaccattgtgtgtgcttaccaggcttccatgggcgtgactgcgagcgcaaggctggaccctgtgaacaggcaggctccccatgccgcaatggcgggcagtgccaggacgaccagggctttgctctcaacttcacgtgccgctgcttggtgggctttgtgggtgcccgctgtgaggtaaatgtggatgactgcctgatgcggccttgtgctaacggtgccacctgccttgacggcataaaccgcttctcctgcctctgtcctgagggctttgctggacgcttctgcaccatcaacctggatgactgtgccagccgcccatgccagagaggggcccgctgtcgggaccgtgtccacgacttcgactgcctctgccccagtggctatggtggcaagacctgtgagcttgtcttacctgtcccagaccccccaaccacagtggacacccctctagggcccacctcagctgtagtggtacctgccacggggccagccccccacagcgcaggggctggtctgctgcggatctcagtgaaggaggtggtgcggaggcaagaggctgggctaggtgagcctagcttggtggccctggtggtgtttggggccctcactgctgccctggttctggctactgtgttgctgaccctgagggcctggcgccggggtgtctgcccccctggaccctgttgctaccctgccccacactatgctccagcgtgccaggaccaggagtgtcaggttagcatgctgccagcagggctccccctgccacgtgacttgccccctgagcctggaaagaccacagcactgtgatggaggtgggggctttctggcccccttcctcacctcttccacccctcagactggagtggtccgttctcaccacccttcagcttgggtacacacacagaggagacctcagcctcacaccagaaatattatttttttaatacacagaatgtaagatggaattttatcaaataaaactatgaaaatgcaaaaaaaaaaaaaaaa

SEQ ID NO: 16 - protein, 183 aa, Homo sapiens
MRAAYLFLLFLPAGLLAQGQYDLDPLPPFPDHVQYTHYSDQIDNPDYYDYQEVTPRPSEEQFQFQSQQQVQQEVIPAPTPEPGNAELEPTEPGPLDCREEQYPCTRLYSIHRPCKQCLNEVCFYSLRRVYVINKEICVRTVCAHEELLRADLCRDKFSKCGVMASSGLCQSVAASCARSCGSC

SEQ ID NO: 17 - protein, 183 aa, Homo sapiens; identical to SEQ ID NO: 16

SEQ ID NO: 18 - protein, 182 aa, Homo sapiens
MRAAYLFLLFLPGLLAQGQYDLDPLPPFPDHVQYTHYSDQIDNPDYYDYQEVTPRPSEEQFQFQSQQQVQQEVIPAPTPEPGNAELEPTEPGPLDCREEQYPCTRLYSIHRPCKQCLNEVCFYSLRRVYVINKEICVRTVCAHEELLRADLCRDKFSKCGVMASSGLCQSVAASCARSCGSC

SEQ ID NO: 19 - protein, 182 aa, Homo sapiens; identical to SEQ ID NO: 18

SEQ ID NO: 20 - DNA, 1105 bp, Homo sapiens
attgcaacttggtctcacagtggcttaggccagggtgggagcagtgaacggagtcacaaaagaaatttttcagctgtcctctctgacaccaccccggcctgcctctttgttgccatgagagctgcctacctcttcctgctattcctgcctgcaggcttgctggctcagggccagtatgacctggacccgctgccgccgttccctgaccacgtccagtacacccactatagcgaccagatcgacaacccagactactatgattatcaagaggtgactcctcggccctccgaggaacagttccagttccagtcccagcagcaagtccaacaggaagtcatcccagccccaaccccagaaccaggaaatgcagagctggagcccacagagcctgggcctcttgactgccgtgaggaacagtacccgtgcacccgcctctactccatacacaggccttgcaaacagtgtctcaacgaggtctgcttctacagcctccgccgtgtgtacgtcattaacaaggagatctgtgttcgtacagtgtgtgcccatgaggagctcctccgagctgacctctgtcgggacaagttctccaaatgtggcgtgatggccagcagcggcctgtgccaatccgtggcggcctcctgtgccaggagctgtgggagctgctagggtggtgctggcatcctgagtcctggccctcctgggatctggggccctcgggccctgcctgacctggtgcttttttccccatccccatgttccttttattctgtaaaaagttagtggactgcagccctgggggttgcaggctgcggtgcctcaggcccctccttcagcctgtggccacctctggggcacaatgggggctccccactgcccagtctgcccctcgggttgggggagtatcccaggcctctctgtgggacctgggcccctgacgggccttctcagcccgttttgaggacagacagtcccccgaggtaggctacatccccccaccccagctggtctgcttggatttcctacagcccccgtgggcatggaccacctttattttatacaaaattaaaaacaagtttttacaaaaaaaaaaaaaaaaaaaaaaaaa

SEQ ID NO: 21 - DNA, 1124 bp, Homo sapiens
gccccgtcgggggcccggagggggactcggagcgggccaaggggcggctccggcgggcggactcggagcgggcggcggagtgacccggacagctgtcctctctgacaccaccccggcctgcctctttgttgccatgagagctgcctacctcttcctgctattcctgcctgcaggcttgctggctcagggccagtatgacctggacccgctgccgccgttccctgaccacgtccagtacacccactatagcgaccagatcgacaacccagactactatgattatcaagaggtgactcctcggccctccgaggaacagttccagttccagtcccagcagcaagtccaacaggaagtcatcccagccccaaccccagaaccaggaaatgcagagctggagcccacagagcctgggcctcttgactgccgtgaggaacagtacccgtgcacccgcctctactccatacacaggccttgcaaacagtgtctcaacgaggtctgcttctacagcctccgccgtgtgtacgtcattaacaaggagatctgtgttcgtacagtgtgtgcccatgaggagctcctccgagctgacctctgtcgggacaagttctccaaatgtggcgtgatggccagcagcggcctgtgccaatccgtggcggcctcctgtgccaggagctgtgggagctgctagggtggtgctggcatcctgagtcctggccctcctgggatctggggccctcgggccctgcctgacctggtgcttttttccccatccccatgttccttttattctgtaaaaagttagtggactgcagccctgggggttgcaggctgcggtgcctcaggcccctccttcagcctgtggccacctctggggcacaatgggggctccccactgcccagtctgcccctcgggttgggggagtatcccaggcctctctgtgggacctgggcccctgacgggccttctcagcccgttttgaggacagacagtcccccgaggtaggctacatccccccaccccagctggtctgcttggatttcctacagcccccgtgggcatggaccacctttattttatacaaaattaaaaacaagtttttacaaaaaaaaaaaaaaaaaaaaaaaaa

SEQ ID NO: 22 - DNA, 1102 bp, Homo sapiens
attgcaacttggtctcacagtggcttaggccagggtgggagcagtgaacggagtcacaaaagaaatttttcagctgtcctctctgacaccaccccggcctgcctctttgttgccatgagagctgcctacctcttcctgctattcctgcctggcttgctggctcagggccagtatgacctggacccgctgccgccgttccctgaccacgtccagtacacccactatagcgaccagatcgacaacccagactactatgattatcaagaggtgactcctcggccctccgaggaacagttccagttccagtcccagcagcaagtccaacaggaagtcatcccagccccaaccccagaaccaggaaatgcagagctggagcccacagagcctgggcctcttgactgccgtgaggaacagtacccgtgcacccgcctctactccatacacaggccttgcaaacagtgtctcaacgaggtctgcttctacagcctccgccgtgtgtacgtcattaacaaggagatctgtgttcgtacagtgtgtgcccatgaggagctcctccgagctgacctctgtcgggacaagttctccaaatgtggcgtgatggccagcagcggcctgtgccaatccgtggcggcctcctgtgccaggagctgtgggagctgctagggtggtgctggcatcctgagtcctggccctcctgggatctggggccctcgggccctgcctgacctggtgcttttttccccatccccatgttccttttattctgtaaaaagttagtggactgcagccctgggggttgcaggctgcggtgcctcaggcccctccttcagcctgtggccacctctggggcacaatgggggctccccactgcccagtctgcccctcgggttgggggagtatcccaggcctctctgtgggacctgggcccctgacgggccttctcagcccgttttgaggacagacagtcccccgaggtaggctacatccccccaccccagctggtctgcttggatttcctacagcccccgtgggcatggaccacctttattttatacaaaattaaaaacaagtttttacaaaaaaaaaaaaaaaaaaaaaaaaa

SEQ ID NO: 23 - DNA, 1121 bp, Homo sapiens
gccccgtcgggggcccggagggggactcggagcgggccaaggggcggctccggcgggcggactcggagcgggcggcggagtgacccggacagctgtcctctctgacaccaccccggcctgcctctttgttgccatgagagctgcctacctcttcctgctattcctgcctggcttgctggctcagggccagtatgacctggacccgctgccgccgttccctgaccacgtccagtacacccactatagcgaccagatcgacaacccagactactatgattatcaagaggtgactcctcggccctccgaggaacagttccagttccagtcccagcagcaagtccaacaggaagtcatcccagccccaaccccagaaccaggaaatgcagagctggagcccacagagcctgggcctcttgactgccgtgaggaacagtacccgtgcacccgcctctactccatacacaggccttgcaaacagtgtctcaacgaggtctgcttctacagcctccgccgtgtgtacgtcattaacaaggagatctgtgttcgtacagtgtgtgcccatgaggagctcctccgagctgacctctgtcgggacaagttctccaaatgtggcgtgatggccagcagcggcctgtgccaatccgtggcggcctcctgtgccaggagctgtgggagctgctagggtggtgctggcatcctgagtcctggccctcctgggatctggggccctcgggccctgcctgacctggtgcttttttccccatccccatgttccttttattctgtaaaaagttagtggactgcagccctgggggttgcaggctgcggtgcctcaggcccctccttcagcctgtggccacctctggggcacaatgggggctccccactgcccagtctgcccctcgggttgggggagtatcccaggcctctctgtgggacctgggcccctgacgggccttctcagcccgttttgaggacagacagtcccccgaggtaggctacatccccccaccccagctggtctgcttggatttcctacagcccccgtgggcatggaccacctttattttatacaaaattaaaaacaagtttttacaaaaaaaaaaaaaaaaaaaaaaaaa

SEQ ID NO: 24 - protein, 173 aa, Homo sapiens
MSLLGPKVLLFLAAFIITSDWIPLGVNSQRGDDVTQATPETFTEDPNLVNDPATDETVLAVLADIAPSTDDLASLSEKNTTAECWDEKFTCTRLYSVHRPVKQCIHQLCFTSLRRMYIVNKEICSRLVCKEHEAMKDELCRQMAGLPPRRLRRSNYFRLPPCENVDLQRPNGL

SEQ ID NO: 25 - DNA, 2900 bp, Homo sapiens
attccagcctcattgtaacacacattctacgcctagcctggctttcttgctctccctcatctcattgtttcagcggaggccaaatctgaagtcctttccagggagtggctctgttcatcttattcgccagccaaagtaggaacagcgtaagaggagagagacacattcagcagccaaaggactcggtggaaagagcagaacaccatagacaatatgtcgctcttgggacccaaggtgctgctgtttcttgctgcattcatcatcacctctgactggatacccctgggggtcaatagtcaacgaggagacgatgtgactcaagcgactccagaaacattcacagaagatcctaatctggtgaatgatcccgctacagatgaaacagttttggctgttttggctgatattgcaccttccacagatgacttggcctccctcagtgaaaaaaataccactgcagagtgctgggatgagaaatttacctgcacaaggctctactctgtgcatcggccggttaaacaatgcattcatcagttatgcttcaccagtttacgacgtatgtacatcgtcaacaaggagatctgctctcgtcttgtctgtaaggaacacgaagctatgaaagatgagctttgccgtcagatggctggtctgccccctaggagactccgtcgctccaattacttccgacttcctccctgtgaaaatgtggatttgcagagacccaatggtctgtgatcattgaaaaagaggaaagaagaaaaaatgtatgggtgagaggaaggaggatctccttcttctccaaccattgacagctaacccttagacagtatttcttaaaccaatccttttgcaatgtccagcttttacccctactctctactttttcacccaaactgataacatttatctcattttctagcacttaaaatacaaagtctatattattgcataattttgctgcttctcaatatcatagacacagtgaatagatgatgactatatggcttatatacaaacattctatgtacaatttcaagggagactaaactttaggctaataatctttactattgaatctgtctgatatagatcttagggttgaagaagctatctttgtctatttgggctaaccatagaatttcatttattttcctcacaatattttcctagaccaactccccatcattcacgtgttcctctttactcttactttaactattttgctggcttgcccgaaaatttgcctggcaagtcttccttataagacacatcatggtaagttttgtagtcctgtaagattctgcaacacagtcaagaattatacaatcctactagcaatatataaggacccaaaatgtcttctgctaagctcagaggctggggctaaagcatgaggactatgccagctatagaacttggactcataattcgctatccaatttttcatgcagttgtctagtcgggaagtaaggttggaaactaagtctcatttactgattcgtttatgggtagtaccgggatgaacccaccaccacaaagcaaattagacaacttaatgtgaaatcataccattggttgacgtttccttgagttgctacttcgttcatcttcacaacttaacaagtgcacggtcgaattattgtgcaagtggcttttggatatcctgattggggcctaagaagggcattcagacttgaattttaataggcagacagaaagtttgcctaatagttaatacgaaagagtgaaagaaacacaatattcagacaacccacattcttatcctggctctagcagtaaccacgtagccttggataagccattttccttcattaggtcctggtttaatttcctcatctttaaaatgagaaggttaaatttatcttagtactgctgggcgcagtggctcatgcctgtaatctgagcactttgggaagctgaggcgggtggatcacttgaggtcagaaatttgagacgagcctggccaacatggtgaaaccccatctctactaaaaatacaaaaattagctgggcgtggtggcacgtgcctgtaatcccagctactcgggaggctgaggcaggagaatcaattgaacctgggaggcagaggttgcagtgagccgagatggcgccattgcactccagcctgggtgacaaaagcaaaagtccatcttaagaaatatatatatatattatatatattcttagttctaagatttcctttaattctatgattctctggatttaaatgcattattcatatttcttgaagcttagatacagtctaattcatagcaaccatatctgctttatcctaggtgagggtagcagtccacaatggaatagaagaaaatcccattataacaaatgacaaattatatatcatgaatccttctgtctgactaactcaataactttctataaaagccaatggaattcaaataggagctaggagacaacaagttatatatgacagtggaggttgtattccttttatattgctgagaaaactagttaaatgatcagattcttgctgttaagaaacaatttcgtttaatgggatctgtacaactgattttaaaaaaatgctacaaaaagccccaaagcatataatctctactccttacagtctctagaattaaatgtactcatttagacaacatattaaatgcatattttagccactttagagaaacctcataggcacagagtttccaagattaattttaagaatatcttcacgaacttgaccctcctactccacattgcaacatttccatcagacagcatttcaattccagtattatgtatattgcaaattaaacattttaaaatatttttttccaatttatttctcaaaataaaatgtcttttgttctggtaaaaaaaaaaaaaaaaaa

SEQ ID NO: 26 - DNA, 5988 bp, Homo sapiens
ctgcccggcgtgctgggtagaggtggccagccccggccgctgctgccagacgggctctccgggtccttctccgagagccgggcgggcacgcgtcattgtgttacctgcggccggcccgcgagctaggctggtttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtacggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattatgagaacaagaactccaaaatgtctaaaataaggacacacaattctgaagtagaagaggacgacatggacaaacaccagcagaaagcccggtttgccaagcagccggcgtacacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttattttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaatttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatcttactttgtaaattctgatttcttttcaccattcgtacataatactgaaccacttgtagatttgatttttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaacatggaacagtgtggcctcttctgagcttacgtagttctaccggctttgccatgtgcttctgccaccctgctgagtctgttctggtaatcggggtataataggctctgcctgacagagggatggaggaagaactgaaaggcttttcaaccacaaaactcatctggagttctcaaagacctggggctgctgtgaagctggaactgcgggagccccatctaggggagccttgattcccttgttattcaacagcaagtgtgaatactgcttgaataaacaccactggattaatggcca

SEQ ID NO: 27 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 28 - DNA, 5845 bp, Homo sapiens
ctcatgcatatgcaggtgcgcgggtgacgaatgggcgagcgagctgtcagtctcgttccgaacttgttggctgcggtgccgggagcgcgggcgcgcagagccgaggccgggacccgctgccttcaccgccgccgccgtcgccgccgggtgggagccgggccgggcagccggagcgcggccgccagcgagccggagctgccgccgcccctgcacgcccgccgcccaggcccgcgcgccgcggcgctgcgctcgaccccgcccgcgccgccgccgccgccgcctctgccgctgccgctgcctctgcgggcgctcggagggcgggcgggcgctgggaggccggcgcggcggctgggagccgggcgcgggcggcggcggcggggccgggcgggcgggtcgcgggggcaatgcgggcgcagggccgggggcgccttccccggcggctgctgctgctgctggcgctctgggtgcaggcggcgcggcccatgggctatttcgagctgcagctgagcgcgctgcggaacgtgaacggggagctgctgagcggcgcctgctgtgacggcgacggccggacaacgcgcgcggggggctgcggccacgacgagtgcgacacgtacgtgcgcgtgtgccttaaggagtaccaggccaaggtgacgcccacggggccctgcagctacggccacggcgccacgcccgtgctgggcggcaactccttctacctgccgccggcgggcgctgcgggggaccgagcgcgggcgcgggcccgggccggcggcgaccaggacccgggcctcgtcgtcatccccttccagttcgcctggccgcgctcctttaccctcatcgtggaggcctgggactgggacaacgataccaccccgaatgaggagctgctgatcgagcgagtgtcgcatgccggcatgatcaacccggaggaccgctggaagagcctgcacttcagcggccacgtggcgcacctggagctgcagatccgcgtgcgctgcgacgagaactactacagcgccacttgcaacaagttctgccggccccgcaacgactttttcggccactacacctgcgaccagtacggcaacaaggcctgcatggacggctggatgggcaaggagtgcaaggaagctgtgtgtaaacaagggtgtaatttgctccacgggggatgcaccgtgcctggggagtgcaggtgcagctacggctggcaagggaggttctgcgatgagtgtgtcccctaccccggctgcgtgcatggcagttgtgtggagccctggcagtgcaactgtgagaccaactggggcggcctgctctgtgacaaagacctgaactactgtggcagccaccacccctgcaccaacggaggcacgtgcatcaacgccgagcctgaccagtaccgctgcacctgccctgacggctactcgggcaggaactgtgagaaggctgagcacgcctgcacctccaacccgtgtgccaacgggggctcttgccatgaggtgccgtccggcttcgaatgccactgcccatcgggctggagcgggcccacctgtgcccttgacatcgatgagtgtgcttcgaacccgtgtgcggccggtggcacctgtgtggaccaggtggacggctttgagtgcatctgccccgagcagtgggtgggggccacctgccagctggacgccaatgagtgtgaagggaagccatgccttaacgctttttcttgcaaaaacctgattggcggctattactgtgattgcatcccgggctggaagggcatcaactgccatatcaacgtcaacgactgtcgcgggcagtgtcagcatgggggcacctgcaaggacctggtgaacgggtaccagtgtgtgtgcccacggggcttcggaggccggcattgcgagctggaacgagacgagtgtgccagcagcccctgccacagcggcggcctctgcgaggacctggccgacggcttccactgccactgcccccagggcttctccgggcctctctgtgaggtggatgtcgacctttgtgagccaagcccctgccggaacggcgctcgctgctataacctggagggtgactattactgcgcctgccctgatgactttggtggcaagaactgctccgtgccccgcgagccgtgccctggcggggcctgcagagtgatcgatggctgcgggtcagacgcggggcctgggatgcctggcacagcagcctccggcgtgtgtggcccccatggacgctgcgtcagccagccagggggcaacttttcctgcatctgtgacagtggctttactggcacctactgccatgagaacattgacgactgcctgggccagccctgccgcaatgggggcacatgcatcgatgaggtggacgccttccgctgcttctgccccagcggctgggagggcgagctctgcgacaccaatcccaacgactgccttcccgatccctgccacagccgcggccgctgctacgacctggtcaatgacttctactgtgcgtgcgacgacggctggaagggcaagacctgccactcacgcgagttccagtgcgatgcctacacctgcagcaacggtggcacctgctacgacagcggcgacaccttccgctgcgcctgcccccccggctggaagggcagcacctgcgccgtcgccaagaacagcagctgcctgcccaacccctgtgtgaatggtggcacctgcgtgggcagcggggcctccttctcctgcatctgccgggacggctgggagggtcgtacttgcactcacaataccaacgactgcaaccctctgccttgctacaatggtggcatctgtgttgacggcgtcaactggttccgctgcgagtgtgcacctggcttcgcggggcctgactgccgcatcaacatcgacgagtgccagtcctcgccctgtgcctacggggccacgtgtgtggatgagatcaacgggtatcgctgtagctgcccacccggccgagccggcccccggtgccaggaagtgatcgggttcgggagatcctgctggtcccggggcactccgttcccacacggaagctcctgggtggaagactgcaacagctgccgctgcctggatggccgccgtgactgcagcaaggtgtggtgcggatggaagccttgtctgctggccggccagcccgaggccctgagcgcccagtgcccactggggcaaaggtgcctggagaaggccccaggccagtgtctgcgaccaccctgtgaggcctggggggagtgcggcgcagaagagccaccgagcaccccctgcctgccacgctccggccacctggacaataactgtgcccgcctcaccttgcatttcaaccgtgaccacgtgccccagggcaccacggtgggcgccatttgctccgggatccgctccctgccagccacaagggctgtggcacgggaccgcctgctggtgttgctttgcgaccgggcgtcctcgggggccagtgctgtggaggtggccgtgtccttcagccctgccagggacctgcctgacagcagcctgatccagggcgcggcccacgccatcgtggccgccatcacccagcgggggaacagctcactgctcctggctgtcaccgaggtcaaggtggagacggttgttacgggcggctcttccacaggtctgctggtgcctgtgctgtgtggtgccttcagcgtgctgtggctggcgtgcgtggtcctgtgcgtgtggtggacacgcaagcgcaggaaagagcgggagaggagccggctgccgcgggaggagagcgccaacaaccagtgggccccgctcaaccccatccgcaaccccattgagcggccggggggccacaaggacgtgctctaccagtgcaagaacttcacgccgccgccgcgcagggcggacgaggcgctgcccgggccggccggccacgcggccgtcagggaggatgaggaggacgaggatctgggccgcggtgaggaggactccctggaggcggagaagttcctctcacacaaattcaccaaagatcctggccgctcgccggggaggccggcccactgggcctcaggccccaaagtggacaaccgcgcggtcaggagcatcaatgaggcccgctacgccggcaaggagtaggggcggctgccagctgggccgggacccagggccctcggtgggagccatgccgtctgccggacccggaggccgaggccatgtgcatagtttctttattttgtgtaaaaaaaccaccaaaaacaaaaaccaaatgtttattttctacgtttctttaaccttgtataaattattcagtaactgtcaggctgaaaacaatggagtattctcggatagttgctatttttgtaaagtttccgtgcgtggcactcgctgtatgaaaggagagagcaaagggtgtctgcgtcgtcaccaaatcgtagcgtttgttaccagaggttgtgcactgtttacagaatcttccttttattcctcactcgggtttctctgtggctccaggccaaagtgccggtgagacccatggctgtgttggtgtggcccatggctgttggtgggacccgtggctgatggtgtggcctgtggctgtcggtgggactcgtggctgtcaatgggacctgtggctgtcggtgggacctacggtggtcggtgggaccctggttattgatgtggccctggctgccggcacggcccgtggctgttgacgcacctgtggttgttagtggggcctgaggtcatcggcgtggcccaaggctggcaggtcaacctcgcgcttgctggccagtccaccctgcctgccgtctgtgcttcctcctgcccagaacgcccgctccagcgatctctccactgtgctttcagaagtgcccttcctgctgcgcagttctcccatcctgggacggcggcagtattgaagctcgtgacaagtgccttcacacagacccctcgcaactgtccacgcgtgccgtggcaccaggcgctgcccacctgccggccccggccgcccctcctcgtgaaagtgcatttttgtaaatgtgtacatattaaaggaagcactctgtatatttgattgaataatgccaccattccggcctcccttgttctttcggtgctgtcccttttgtattgagagtgaggttgggggagagccacgccggcagagaggcttggggcagtggggcgcgtgctgggtattggcccacgtggctgtggtggctgtagagggcgagacggttctgttgagtcggggcctgccagggcctcgaatgcgttggcatgccaaggtggtggatgcaggtttggccaaaaccttcctgggaatggggaggggggtgtctaggtgcctggcacccgaccctgactaaaacagctgaaaacagttttataaaatagtataaaattgcttacccacgaatacgtatcaaggtcttaaggaattaacaggtcaaaatttttattcattcatttattcctttgttttgcttggtcattcagaggcaaggtcagcatttgatggaggcaggagagaagcagcccttccacctggccctggagctggttttgcccctcactgcttgagccaactggaacgtggcatggtggcctcagggctgggcatggaaggccgactccaaggctgactccgagccgcctgtcagtgatgtgcaagtctttaccctgctgtgaggggtaggaggtcaactgagtcaaggagcaaagccaagaaccagatctgtacaaaagagaaaaaaaaagtcctgctgacagcctgggagtgaggctgccttgcacaggcagaacaaatgaataaatgaatgaacgaataaaaattttgacctggtaaaaaaaaaaaa

SEQ ID NO: 29 - protein, 1238 aa, Homo sapiens
MRAQGRGRLPRRLLLLLALWVQAARPMGYFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCEKAEHACTSNPCANGGSCHEVPSGFECHCPSGWSGPTCALDIDECASNPCAAGGTCVDQVDGFECICPEQWVGATCQLDANECEGKPCLNAFSCKNLIGGYYCDCIPGWKGINCHINVNDCRGQCQHGGTCKDLVNGYQCVCPRGFGGRHCELERDECASSPCHSGGLCEDLADGFHCHCPQGFSGPLCEVDVDLCEPSPCRNGARCYNLEGDYYCACPDDFGGKNCSVPREPCPGGACRVIDGCGSDAGPGMPGTAASGVCGPHGRCVSQPGGNFSCICDSGFTGTYCHENIDDCLGQPCRNGGTCIDEVDAFRCFCPSGWEGELCDTNPNDCLPDPCHSRGRCYDLVNDFYCACDDGWKGKTCHSREFQCDAYTCSNGGTCYDSGDTFRCACPPGWKGSTCAVAKNSSCLPNPCVNGGTCVGSGASFSCICRDGWEGRTCTHNTNDCNPLPCYNGGICVDGVNWFRCECAPGFAGPDCRINIDECQSSPCAYGATCVDEINGYRCSCPPGRAGPRCQEVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTGLLVPVLCGAFSVLWLACVVLCVWWTRKRRKERERSRLPREESANNQWAPLNPIRNPIERPGGHKDVLYQCKNFTPPPRRADEALPGPAGHAAVREDEEDEDLGRGEEDSLEAEKFLSHKFTKDPGRSPGRPAHWASGPKVDNRAVRSINEARYAGKE

SEQ ID NO: 30 - DNA, 1309 bp, Canis lupus familiaris
cgggagcgggcgcagtgtgcgcgggacgcgagcccccgagcgcgcagaggcgccgccgctgagagccccccggcccgccgcgccgacccgcccgcggagccccggccgcgcagccagcatgaagcgagcccacccggactacagctcctcggacagcgagctggacgagaccgtcgaagtggagaaggagagcgcggatgagaatggaaacttgagttcggctctaggttccatgtccccaactacatcttcacagatcttggccaggaaaagacgaagaggcatcattgagaagcgccgacgggaccggattaataacagtttgtctgagctcaggcggctggtacccagtgcttttgagaagcagggatctgctaagctagaaaaagccgaaatcctgcagatgaccgtggatcacctgaaaatgctgcacacggcaggaggaaaaggctatttcgacgcgcacgcccttgctatggactatcggagtttggggttccgggagtgcctggcggaggtggcccgatatctgagcatcattgagggactggatgcctccgacccgcttcgagttcggctggtctcccaccttaacaactacgcctcccagcgggaagcggccagcggcgcccacgcaggcctgggacacctcccctggggcagcgccttcggacaccacccgcacgtcgcgcaccccctgctgctgccccagagcggccacgggaacactggcaccagcgcctcgcccacggacccgcaccaccagggcaggttggctgcggcgcatccggaggcgcccgccttgcgcgcgccccctagcggcggcctcggaccggtgctccccgtggtcacctcggcctccaagctctcgccgcccctgctgtcctcggtggcttccctgtcggccttccccttctcctttggctcctttcacctcctgtctcccaatgcactgagcccttcggcacccacgcaggccgcaaaccttggcaagccctatagaccttgggggacggagattggagctttttaaagacccgacgctgtaggatggagggaggggaacacctaaaggcccagctgagctgggctgtggccaacatcaccttaaagtcctcagtaaaagtaaagaggagaaaaggtacacgttcagatcaattttgttgaaagactaaaggtttgttggtttactttttcttttttaatgttttttttttcttttttaaaatcaggtcccgtcttagcagtttttaaaagctagttgttaaattttgttccaaacattacattgaaatagtgagtataaacc

SEQ ID NO: 31 - protein, 304 aa, Canis lupus familiaris
MKRAHPDYSSSDSELDETVEVEKESADENGNLSSALGSMSPTTSSQILARKRRRGIIEKRRRDRINNSLSELRRLVPSAFEKQGSAKLEKAEILQMTVDHLKMLHTAGGKGYFDAHALAMDYRSLGFRECLAEVARYLSIIEGLDASDPLRVRLVSHLNNYASQREAASGAHAGLGHLPWGSAFGHHPHVAHPLLLPQSGHGNTGTSASPTDPHHQGRLAAAHPEAPALRAPPSGGLGPVLPVVTSASKLSPPLLSSVASLSAFPFSFGSFHLLSPNALSPSAPTQAANLGKPYRPWGTEIGAF

SEQ ID NO: 32 - DNA, 2319 bp, Homo sapiens
ttccccactcccccgccctccccagggccctgggaaggggctcagcgtgggaaaggatggttgagttttaaccagaggcaaagcgtgagcgggatcagtgtgtgcggaacgcaagcagccgagagcggagaggcgccgctgtagttaactcctccctgcccgccgcgccgaccctccccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatcccagctgggctggactgttgccaacatcaccttaaagtcgtcagtaaaagtaaaaaggaaaaaggtacactttcagataattttttttttaaagactaaaggtttgttggtttacttttatcttttttaatgtttttttcatcatgtcatgtattagcagtttttaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttagtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggccctgaatccatctgaccagctgctggtatctgccaggactggcagttctgatttagttaggagagagccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaaactgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaagaaagaacaaagtaaaaaaaaaaaaaaaaaaaaaaaaaaa

SEQ ID NO: 33 - protein, 304 aa, Homo sapiens
MKRAHPEYSSSDSELDETIEVEKESADENGNLSSALGSMSPTTSSQILARKRRRGIIEKRRRDRINNSLSELRRLVPSAFEKQGSAKLEKAEILQMTVDHLKMLHTAGGKGYFDAHALAMDYRSLGFRECLAEVARYLSIIEGLDASDPLRVRLVSHLNNYASQREAASGAHAGLGHIPWGTVFGHHPHIAHPLLLPQNGHGNAGTTASPTEPHHQGRLGSAHPEAPALRAPPSGSLGPVLPVVTSASKLSPPLLSSVASLSAFPFSFGSFHLLSPNALSPSAPTQAANLGKPYRPWGTEIGAF

SEQ ID NO: 34 - DNA, 2331 bp, Homo sapiens
ttccccactcccccgccctccccagggccctgggaaggggctcagcgtgggaaaggatggttgagttttaaccagaggcaaagcgtgagcgggatcagtgtgtgcggaacgcaagcagccgagagcggagaggcgccgctgtagttaactcctccctgcccgccgcgccgaccctccccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcaggtaatggagcaaggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatcccagctgggctggactgttgccaacatcaccttaaagtcgtcagtaaaagtaaaaaggaaaaaggtacactttcagataattttttttttaaagactaaaggtttgttggtttacttttatcttttttaatgtttttttcatcatgtcatgtattagcagtttttaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttagtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggccctgaatccatctgaccagctgctggtatctgccaggactggcagttctgatttagttaggagagagccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaaactgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaagaaagaacaaagtaaaaaaaaaaaaaaaaaaaaaaaaaaa

SEQ ID NO: 35 - protein, 308 aa, Homo sapiens
MKRAHPEYSSSDSELDETIEVEKESADENGNLSSALGSMSPTTSSQILARKRRRGIIEKRRRDRINNSLSELRRLVPSAFEKQVMEQGSAKLEKAEILQMTVDHLKMLHTAGGKGYFDAHALAMDYRSLGFRECLAEVARYLSIIEGLDASDPLRVRLVSHLNNYASQREAASGAHAGLGHIPWGTVFGHHPHIAHPLLLPQNGHGNAGTTASPTEPHHQGRLGSAHPEAPALRAPPSGSLGPVLPVVTSASKLSPPLLSSVASLSAFPFSFGSFHLLSPNALSPSAPTQAANLGKPYRPWGTEIGAF

SEQ ID NO: 36 - DNA, 4131 bp, Homo sapiens
ccgactgggagccttagccgcggggctgagaccaggcagcctgcgttcgccatgaagcgacccaaggagccgagcggctccgacggggagtccgacggacccatcgacgtgggccaagagggccagctgagccagatggccaggccgctgtccacccccagctcttcgcagatgcaagccaggaagaaacgcagagggatcatagagaaacggcgtcgagaccgcatcaacagtagcctttctgaattgcgacgcttggtccccactgcctttgagaaacagggctcttccaagctggagaaagccgaggtcttgcagatgacggtggatcacttgaaaatgctccatgccactggtgggacaggattctttgatgcccgagccctggcagttgacttccggagcattggttttcgggagtgcctcactgaggtcatcaggtacctgggggtccttgaagggcccagcagccgtgcagaccccgtccggattcgccttctctcccacctcaacagctacgcagccgagatggagccttcgcccacgcccactggccctttggccttccctgcctggccctggtctttcttccatagctgtccagggctgccagccctgagcaaccagctcgccatcctgggaagagtgcccagccctgtcctccccggtgtctcctctcctgcttaccccatcccagccctccgaaccgctccccttcgcagagccacaggcatcatcctgccagcccggaggaatgtgctgcccagtcgaggggcatcttccacccggagggcccgccccctagagaggccagcgacccctgtgcctgtcgcccccagcagcagggctgccaggagcagccacatcgctcccctcctgcagtcttcctccccaacaccccctggtcctacagggtcggctgcttacgtggctgttcccacccccaactcatcctccccagggccagctgggaggccagcgggagccatgctctaccactcctgggtctctgaaatcactgaaatcggggctttctgagctgccccttcaccaccccgccccaaggaataaggaaggttcttttaccaggagcccaaaaaagggcactgccttttctgctttgcttcatggactggctcatatgtgaaggcacgttctccagccatcagaggccccctcctcctccaacccatctctccttctcactgttatcccagcttatccacccagctctcctggagctgttctggtctcagaggcttggttccatttctcacctgaacagatgagtcctgggagagaccctcagagatccgcccagacccctctcctgccctctgcacaccagcagcaggcatgaaccttgggtctgggaaaaagctttaacctgcagggcaccaggacccaaggcaggctgttccttggggcggtcagaccccagtcaggagcaatgactgactggctgcagccttcccacgccaagaggctggaacatagtgtctgcctcgcttcctggagatagtaactgagcaggggctacaaagaggtctcctgggaaccctgtctgccccttcccacctgtccttgggccacaccatcacactgaaccacaggacagaccctttctccaccacagccaaggcctggagactgggggcccagcagagcctgctcccaccctcctcccagcagcagacacccaccctctcactgactaacaggtccctgcacacagctggcctggtaaacccagctgggaggtttctaggcagcagcaaaactctgtgacagggtgtcctcacaccaggccttggacagctctcccagacaggagccagggttgagcaatggagagcccagcccccacgtcttacagtcgccatcctccaggcgtgtggtccctccccattgggtgcacagtgcagaggggccgtggccccatgtgatggtgcgcagagaggaacctcttgggattcagcaccagacgtctgtgctgcctggtttgcatccggctcacagagcccagactgctggaacagccaaggactgtcaggctggacaaaaataactgcaaggaggggcaagagaaaggatgattcgaggcaccttggcccttcaaggtcatgcagtgggtcgagcgcctgagatcctgttcaccaggactccacagagctggctctgctcagaagccatttcattccccggctccaccctaggccactttttctaacagaggaaacaaatggtccagcagtcgttcccagcagaacagcggagcctggactgacacccagtgggaccagtgttgccacaccagttgataaaatgcagaaacccttctgtactcgttggtaaatatctactcccccaagtgactccaggtgccccccaccgcctggcacttcccccaggactcctacgatctggttactgcctggccgatccaaggctgtggagtcccagagccagcagttcactggtgctcattccacactggttagatacttcagttgtcacccctgggaagattctcccacctcctccctttgatggaaccaccctccccagaggctgcattgaggagactccacagactgaaaagtgagtttgcagaaaccttggggaaaagggccctttcaaagaagtggataagagggaggagatcattgagtgacccagaaagctcttttgaaaagacagactcctcaaggagagataaagaggaaagcacctctttcattttttagtgtgagctaattccatcagactgctgtcctcctggacccatctgagatgtgcagtagcaaggagaggggggatcattttagagagtgggtcattggcagggagtgctccggagggaggcagaggggagactgtggtagaaggaagacagaactcacacatgctcccaggattggggacagggacagaggaggtaacagaaggcaaaggccagtttccccgttatcatgaaggggcccactcaggacaggaacaaggacaactcctcctcctcctcctcctctcctgctgctcctgggataccaggtcagtgatgtagtcttgcagtttggcaacttcctagcctgagaatccctagtggggctgtgggaaacacatttccacgttgcaagcatgcaactccaaagaatctgtgatgccactgaaatgagatgggaatgatccagctctttcagcatcttggttgaacttgctttcattgtccctgggatattgtggaaggaaaggtgactgtgtgatctgattctgtggtcaaggacttgcatcttgtgtttctatccccaagccttcctggtgtctccaactcctaccccattgcatgggttgttgcggacatccaataaagatttttttagtgcttctggaaacttccagtagattctacttctaaactatctctggagtccatccacttctgtctgcacccacagccatcctggccaggccacatcacctcccccagatcactgccctggcctcagaaaggtcttccctcttgctttgtcaatcagttctcagtagcagcagagagaaattgaaagctgcaggtcatatcgtatcatctttagtttgaaaacctcactctcttaccctattgttctaaaggtcttcttttggtcccaacctcatttccagcctcatttcttgccagtcccagacttgctccctgagcttctgccacctgcccttccttcatttcctcgacattccagccttgttcccacctccagcactttgcatatgctgttccctttgccaagaatgctcttcccctaccctgtgcatggctgagttctgcagaccctcaggccttggcttcaacgttgcctcgtccaagaggccttcctcgactactttacttgtggagttcctctatcacaaggcctctgttctttcccttcatggagaatttgccactgcatatccatttgtgtaatttacttgttggttgactgtgcctcccactcgagtgtaagctcatgaggccaggtgccatgcctggttcagtctccactctgtacccagcattgagcacagggcctggtccatagttggcgttcaataaatacttgttgaagaagtgaactgaaaaaaaaaaaaaaaaaa

SEQ ID NO: 37 - protein, 328 aa, Homo sapiens
MKRPKEPSGSDGESDGPIDVGQEGQLSQMARPLSTPSSSQMQARKKRRGIIEKRRRDRINSSLSELRRLVPTAFEKQGSSKLEKAEVLQMTVDHLKMLHATGGTGFFDARALAVDFRSIGFRECLTEVIRYLGVLEGPSSRADPVRIRLLSHLNSYAAEMEPSPTPTGPLAFPAWPWSFFHSCPGLPALSNQLAILGRVPSPVLPGVSSPAYPIPALRTAPLRRATGIILPARRNVLPSRGASSTRRARPLERPATPVPVAPSSRAARSSHIAPLLQSSSPTPPGPTGSAAYVAVPTPNSSSPGPAGRPAGAMLYHSWVSEITEIGAF

SEQ ID NO: 38 - DNA, 5795 bp, Homo sapiens
gagcagcggcagcagcagcggaccccggcggcggcggcggcgcgcggtcccagccaggcggccccggtgtcccggccccggtggatgcacggctggggaggagcccatgggccggagctgaggctgcccggggcggcggggcgcggggcagggggcgcggtcgaggcccggaggcggcggcgcaggaggaagcggaggaggtcgggcgctcggggcccgggaggcgggccgcgcagcgccgcagccccgggctcgccatgctcctggcctcggccgtggtggtctgggaatggctgaacgagcacggccgctggcgtccctacagcccagcggtgagccaccacatcgaggcggtggtccgcgccggcccccgcgcggggggcagcgtggtgctgggccaggtggacagccgtctcgcgccctacatcatcgacctgcagtccatgaaccagttccgccaagacacgggaactctccgcccagttcgccgcaactactacgacccctcctcggcccctgggaagggcgtggtgtgggagtgggagaacgacaatggctcctggacgccctacgacatggaagtgggcatcaccatccagcatgcctatgagaagcagcacccctggatcgacctcacttccattggctttagctacgtaattgacttcaacaccatgggccagatcaaccgtcagacccagcgccaacgccgcgtccgccggcgcctcgacctcatctaccccatggtcacagggaccttgcctaaggctcagtcctggccagtcagccctgggccagccacctcgccccccatgtccccctgctcctgtccccagtgtgtcttggtgatgagtgttaaggcagccgtggtcaatggcagcactgggcccctacagctgccagtgacccgcaagaacatgccgcctcctggagtggtcaagctacccccactgccaggctctggggccaagccactggacagcacaggcaccattcgaggcccactgaagaccgccccatcgcaggtgatccggagacaagcctccagcatgcccactgggacaaccatgggctctcctgccagtcccccaggacccaacagcaagaccggaagggtggccctggccaccttgaatcgtaccaacctgcagcgactggccattgcccagtcccgggtgctgatcgcctctggggtccccacagtcccagtgaagaacctaaatgggtccagtcctgtcaaccctgccttggcaggaatcactgggatcctcatgagtgcagcggggctgcctgtgtgtctcaccaggccaccaaagctggtcctacacccaccccccgtcagcaagagtgaaataaaatccatcccaggggtttccaacacaagccgcaagaccaccaaaaaacaagccaagaaaggtaaaaccccagaggaagtgctaaaaaaatatctacagaaagtccggcacccaccagatgaggactgcaccatctgtatggaacgcctcacggccccctcaggctacaagggcccgcagcctacggtaaaacctgacctggtagggaagctgtccagatgcggccacgtctaccacatctactgcttggttgccatgtacaacaatgggaacaaggatggaagtttgcagtgtccaacctgcaagaccatttatggggtgaagacaggcacccaacctccagggaagatggagtaccacctcatcccccactccttgcctggccacccagactgcaaaaccatccggatcatctacagcatcccccccggcattcagggaccggaacacccgaatcctgggaagagtttcagcgcccgaggcttcccacgacactgttaccttccggacagcgagaaagggagaaaagttctgaagctgctgctcgtggcctgggatcgccgcctcatttttgccattggcacctccagcaccacaggcgagtcagacaccgtcatctggaatgaggtccaccacaagacagagtttggctctaatctcactggccatggctacccagatgccaattacctggataatgtgctggctgaactggctgcccagggcatctctgaggacagcactgcccaggagaaggactgaggccagaaaagctttgaggtgggaggggccatggagactgcaggacaggaagtgaggagagtgagtcaatgtagaagaagttggtgtcctgccctcccaactttctatcctcccctcctgccctgtgtccatccctcatccctcccaaccacagtgggagccagactgaatatagcgacatcattcataaatctcatccaacacaaagggagatgggatgagggccatcctgggtctgttcccatggagtttttggtgctgggtaggcaggaatcccctccctaccccacctcccaagtaggggcatggtcagcacacctagggtatgggcagtgcttaggcactccatatcctggctttgggaagccggggtttcttgcctcagccggcttcttgctacttccactctgctttgagactggagtttctgctattctccctctgctggaggcagggagctctcactgtgcaaggttggggggtgggcaaaggggtgaatcactaaactgctgtgacatcagaaactgatgccttggtgtagagcaaggaagcacttcttcccaagagggtcggagaaggaaaagcctctgggagcacattctgctgtcatcacagtccttggcttctctgggccctcctctcctcctcacagctctcacctgtccaaagaggcatctggttctctcatgtggatggatggactctggggttcctctttggagtggcatcccatgatgctgtttctagaccctctctgatcaaaccagagcctgcatcccactgagcatctgaactgtcctcagggagaggagcccacagccttcttcccaactcattctagaccagctcaaagattccatgagtttcatcgagtcactgtgagtggagcccatgctgggctctgtgccctctgtgtctgtgcatgcgcgtgtgtgtgtgggcgtgtgtgcattgctgggccagcttgaagggaaggcccgtcatgtccctgcactctgttttgcaagatgccaaaccccagttctgatggggctccaacagccaggctgtggtcctttgacgttcctcacctgttgccaacctatcccgtagtgaactgaaaccccaatgaagacagaactgtgcctggggagatgcaatgaggtgagggctgaactcatccttttatatttcttttcaagattggatcagagctcatctccatccagtcttgtttctatgaaggcttcaatctgtttccatgcaaatttgctaatcagagcccagagctgctgggtccctcatctccctcatctattatagattgacttacagcagggagagaatctctttagctcattcctaatggagttgggatcacaatatggtctggtccaatctgcatcttgttgtgtcccaagaccctatctcctccccaacattcttattgcctttggctcccagtaaggaacgaattgggggccagggaggagaacaggggggatcaagaagggaaacccaattccccctttgaaagtgggttctttgaactatgtgtttgggggaagttcctctggatactaatttgaatttatatacctcatgttttgggggtttgacgtatatatatatatatatatatgcatatatatttcataatatttggaaggtttttgatgctagaaaaatggaaacaagagaaccttcaaaaatggtacttagatgggaactggaggccaatctttcataaagccagccccatagctgcttgctgttaggcctccagccattttgacattggggtggatagtcgattcacctgcctgtcagtcgattcacctgcctgtcacccagttctgtggatgtgctggtgctgagcctttgctctctttccaaatggttacagggatgttgatcagctccaccagagggagctctgatgggaggaattgctctgccatccttgtccctgtgtctcctgtcggcaggcagccattgtatctcaccagcagaccaggagactggtcccaaggttactgcaccacagggcaatttcctgccatagttaggaaggaaacacctgaactaaatggaagagacatccctgcggtgtttaatatcacacccatgccctttgtcaggttaccatgtacagagattacttggagagcctcatgccgtctctaccttcgcacactggtcaagtatctgctgagcttcttggccgcaaggatgcagaaataggctgagggtccatgggaagaaagacacaatgaggcagtaggaggtggggaagaaaagaagacagactttcaaaatggaattaggcactggggagagatcagtttccccacatcagggagaagaaggtataggtggggaagggggtggccaggagcagaaggaagaagactcaagatggaaagggagccgctgtgcctgtggcaataccacttggagaggtcgacttcataccttcaagccttttcccctgggcttttgattgtgtctgtgccccctttcttgtcctctctgcagatgcccagtaggggctacctcatcctcgtgctgttcttgtgtggctttctgggcagtagggatcttgaatttcctttctaacactgtgcccggcaaggcggggagcattcctctgccctttgtcttgtgccaacctggaaaggtgcagtctagatttcagtgagaaccctgccagctgagccctgtgcatctactaccttgacacagagtgttttcccactagaagctctgctctgctctcctggcccaagtaggggattccatgccttccctttcatggtcttagcaccagcagcctagtttctcccttccagagtctccagggatgacaaattggattggagacaaacctcgtcagatgctcatcccctaaaaggttaattgtgtatttgtggctgcgtgtgcctttgtgttttcattctcttcccatttttgtacattttggtcttctctgtggttttatacttggtcaaaagtactcgtcttggtattgcactgttgtgtgcatgagaaaactgggggaaggctcactggtacaagaaaggacccctgacccctttccttctctgtggtccccggcattagattgggggttctgggagaggcaggtgaatgtcctaagtgaattgttctgtttgtaactggaatgtttttgaagtctttggtgttgctccgtgaaaggacatcgccacctggtgctcatgaggtgtctttgcagaacaataaatggcaaatgaacaaccacaaaattgttactcttgttggccttctgctgtttgtagattagtgcacctatctgtgagggatttgggttacctccctgagtctgtaagcaaccacaagccctgccactgggtgggggaagtccctccccaaccacttaaaaacaaattttccacatattacccacccacacatttgacctggctagactttgtttgcctaaaggaacagaccacattgctgggaaaatgagtaagtgaacgtgtgggagaaaaacacttttagaatcacgaatattcacttttaaaggtctctttgcctggctgcaatatagtgtgtgtttaaattatttacaggctgttgtttctcaaataaatgtttaatattaatcattcccaaactgacaagaacacaaaaataaaatgcaaatacagagcc

SEQ ID NO: 39 - protein, 619 aa, Homo sapiens
MLLASAVVVWEWLNEHGRWRPYSPAVSHHIEAVVRAGPRAGGSVVLGQVDSRLAPYIIDLQSMNQFRQDTGTLRPVRRNYYDPSSAPGKGVVWEWENDNGSWTPYDMEVGITIQHAYEKQHPWIDLTSIGFSYVIDFNTMGQINRQTQRQRRVRRRLDLIYPMVTGTLPKAQSWPVSPGPATSPPMSPCSCPQCVLVMSVKAAVVNGSTGPLQLPVTRKNMPPPGVVKLPPLPGSGAKPLDSTGTIRGPLKTAPSQVIRRQASSMPTGTTMGSPASPPGPNSKTGRVALATLNRTNLQRLAIAQSRVLIASGVPTVPVKNLNGSSPVNPALAGITGILMSAAGLPVCLTRPPKLVLHPPPVSKSEIKSIPGVSNTSRKTTKKQAKKGKTPEEVLKKYLQKVRHPPDEDCTICMERLTAPSGYKGPQPTVKPDLVGKLSRCGHVYHIYCLVAMYNNGNKDGSLQCPTCKTIYGVKTGTQPPGKMEYHLIPHSLPGHPDCKTIRIIYSIPPGIQGPEHPNPGKSFSARGFPRHCYLPDSEKGRKVLKLLLVAWDRRLIFAIGTSSTTGESDTVIWNEVHHKTEFGSNLTGHGYPDANYLDNVLAELAAQGISEDSTAQEKD

SEQ ID NO: 40 - DNA, 2271 bp, Homo sapiens
gaaagctggtgtggagggagaagcgagtgtggtccggagaaagaaggcgtggagaagagggagggagcgagagcgagagaataaatatataaataaatacgagaacgaaatccactccgcagtctccgggctcggaaactttggccccgagcgccagagcgccagagcgcgagagcgcggcgctcgccactctgaggctggcggcctcgattccggccgcgttcccccggcccccctccgccgcggggcctggtctccgggttctgccaggcgcatcagcccgcacaacttctggccgaggccagccggcagaggcggacttggggttggagtgtttgtttgtttgaacttcctcgtcgtcgccaccttccctccccccaacctccaccccacctcacccccctccccagcttctggacgcgtttgactgcagccaggggtggggggtgggggtagggagtgtgtgtggaggggagggagaagaggttaaaaaaaagaagacgaagaagacggaaagaaagagatcgcagcaggggtgaagggagcggacgggaagcgatttttgccgactttggattcgtccccggcgtgcgcaagaatggcggcccttcccggcacggtaccgagaatgatgcggccggctccggggcagaactacccccgcacgggattccctttggaagtgtccaccccgcttggccaaggccgggtcaatcagctgggaggggtcttcatcaatgggcgacccctgcctaaccacatccgccacaagatagtggagatggcccaccatggcatccggccctgtgtcatctcccgacagctgcgtgtctcccacggctgcgtctccaagattctttgccgctaccaggagaccgggtccatccggcctggggccatcggcggcagcaagcccagacaggtggcgactccggatgtagagaaaaagattgaggagtacaagagggaaaacccaggcatgttcagctgggagatccgggacaggctgctgaaggatgggcactgtgaccgaagcactgtgccctcaggtttagtgagttcgattagccgcgtgctcagaatcaagttcgggaagaaagaggaggaggatgaagcggacaagaaggaggacgacggcgaaaagaaggccaaacacagcatcgacggcatcctgggcgacaaagggaaccggctggacgagggctcggatgtggagtcggaacctgacctcccactgaagcgcaagcagcgacgcagtcggaccacattcacggccgagcagctggaggagctggagaaggcctttgagaggacccactacccagacatatacacccgcgaggagctggcgcagaggaccaagctgacagaggcgcgtgtgcaggtctggttcagtaaccgccgcgcccgttggcgtaagcaggcaggagccaaccagctggcggcgttcaaccaccttctgccaggaggcttcccacccaccggcatgcccacgctgcccccctaccagctgccggactccacctaccccaccaccaccatctcccaagatgggggcagcactgtgcaccggcctcagcccctgccaccgtccaccatgcaccagggcgggctggctgcagcggctgcagccgccgacaccagctctgcctacggagcccgccacagcttctccagctactctgacagcttcatgaatccggcggcgccctccaaccacatgaacccggtcagcaacggcctgtctcctcaggtgatgagcatcttgggcaaccccagtgcggtgcccccgcagccacaggctgacttctccatctccccgctgcatggcggcctggactcggccacctccatctcagccagctgcagccagcgggccgactccatcaagccaggagacagcctgcccacctcccaggcctactgcccacccacctacagcaccaccggctacagcgtggaccccgtggccggctatcagtacggccagtacggccagagtgagtgcctggtgccctgggcgtcccccgtccccattccttctcccacccccagggcctcctgcttgtttatggagagctacaaggtggtgtcagggtggggaatgtccatttcacagatggaaaaattgaagtccagccagatggaacagttcacctaaaatgacactgagttgggcaaaacccaggacatctcctggctaagcctctgcttccgtactatggctccaacagaaataaaatacacaacacaaatatcaaaaaaaaaaaa

SEQ ID NO: 41 - protein, 520 aa, Homo sapiens
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSGLVSSISRVLRIKFGKKEEEDEADKKEDDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHSFSSYSDSFMNPAAPSNHMNPVSNGLSPQVMSILGNPSAVPPQPQADFSISPLHGGLDSATSISASCSQRADSIKPGDSLPTSQAYCPPTYSTTGYSVDPVAGYQYGQYGQSECLVPWASPVPIPSPTPRASCLFMESYKVVSGWGMSISQMEKLKSSQMEQFT

SEQ ID NO: 42 - DNA, 2075 bp, Homo sapiens
acaaccccctcttctttgcccggggtggtttgttccaaggagtacagatagccttttcaaaaggcgcagcttaccgcggtgcgcgcggattctggacttgggcgccaactcgtagtccacgctccccggggtcagcagaggggcgctcacgctctcgccacccacctcgctttctcaccccgcgcttcccggcctgggtttttagtcttccttggagcgctctctggcctccgcctccgccagggagcggaaggcggagacagcgagactggccaggggggaggaaagaggacgcgtgtgggcaagggggacaacgggatgtccacgggctcggtgagtgatccggaggagatggagcttcgggggctgcagcgggagtacccggtccccgcctccaagaggccgcccctccgcggcgtagagcgcagctacgcctcgcccagtgacaactcgtcggcagaggaggaggaccccgacggcgaggaggagcgctgcgctctgggcacagccggcagcgcggaaggctgcaagaggaagcggccccgtgtggctgggggcggcggcgcaggtggtagcgcgggcggtggtggcaagaagcccctcccggccaagggctcagccgcagagtgcaagcagtcgcagcggaacgcggccaacgcccgtgagcgtgcccggatgcgcgtgctgagcaaagccttctccaggctcaagaccagcctgccctgggtgccccccgacactaagctctccaagctggacacgctccggctggcttccagttacatcgctcacctgcggcagctgttgcaggaggaccgctatgagaacggctacgtgcacccagtgaacctgacatggccattcgtggtctcgggaagaccggactctgacaccaaagaagtttccgcagccaacagactatgtggaaccaccgcttaaatcggactggaactcacttgatgggattattcgttaaatgcgagtgtttgggggccacggagagaagggagagctcgtgagatgggaagaagtttccgctggattctccttgacccttcccctttccctggaactgtgatcgtgacaggtggcgggtgtggctgtcactgcacagcgcccacggctacagctgcgccggatctgggcgaccacgttttgcctctccaaaaagagcttcctttcgtgacgagacgcggacgcaggtccaccctcgggccctagctctgtagactaactctcggctgctgccccagcccgcgccagacagcccacggatccgttctcagcggagccagattcatcgcacacgtgcgggacggttccacacagccccggcctttcgcggtgacacaatggttagggaacggttagaacgcgctctacatccgctttaaagacagaggtctagacgtgagatccgcgtcgggacagggttttaagtgacaaagaagggcgagtggcttctctgggccgggttcgtactccagcacagcgcccttctaacgggcgggaggaaggccgctgctcgcagggctaggtggagacacacttcccagatcaccgcaggcgggttttacccggagagctctgggccgttcggcctccctgccgggtggcttcttcaatcccgtctccttcccaagctcccggctttttctaatcaggcaggcgtctgtcaaccctctccacttctgggctgaagcctccccaagccccgctgcgccaacctgtgtggggtcttcttcgggcctccctctccgccccgctcctgctcctacctgcagcacccccagctccgactccagactctctgcatcaggtctccccactccacgctccgggcgccccaactccaacaccacgtcctgccgcgcaggttcttccccgcgcggaggagcgcgcagggtgggcggcttaccatagcaagtgatcctgcgatagggaacgcgcccttgccccgaggctgcactaccacaggaaataacatatgtaaataaatttatttttttatgaataataaaacgcgctgtaaaaaccgtgtgccccttggaggtgtcaaaaaaaaaaaaaaaaaa

SEQ ID NO: 43 - protein, 206 aa, Homo sapiens
MSTGSVSDPEEMELRGLQREYPVPASKRPPLRGVERSYASPSDNSSAEEEDPDGEEERCALGTAGSAEGCKRKRPRVAGGGGAGGSAGGGGKKPLPAKGSAAECKQSQRNAANARERARMRVLSKAFSRLKTSLPWVPPDTKLSKLDTLRLASSYIAHLRQLLQEDRYENGYVHPVNLTWPFVVSGRPDSDTKEVSAANRLCGTTA

SEQ ID NO: 44 - DNA, 1520 bp, Homo sapiens
tctgcccttgttaattaccggagcgacagactagggagctccgcccgggatttgcccatcggcggaggcgccaggctcccgtttctccccatccctctcgctgccgtccaggtgcaccgcctgcctctcagcaggatggacgtgatggatggctgccagttctcaccttctgagtacttctacgacggctcctgcataccgtcccccgagggtgaatttggggacgagtttgtgccgcgagtggctgccttcggagcgcacaaagcagagctgcagggctcagatgaggacgagcacgtgcgagcgcctaccggccaccaccaggctggtcactgcctcatgtgggcctgcaaagcctgcaagaggaagtccaccaccatggatcggcggaaggcagccactatgcgcgagcggaggcgcctgaagaaggtcaaccaggctttcgaaaccctcaagaggtgtaccacgaccaaccccaaccagaggctgcccaaggtggagatcctcaggaatgccatccgctacatcgagagcctgcaggagttgctgagagagcaggtggagaactactatagcctgccgggacagagctgctcggagcccaccagccccacctccaactgctctgatggcatgcccgaatgtaacagtcctgtctggtccagaaagagcagtacttttgacagcatctactgtcctgatgtatcaaatgtatatgccacagataaaaactccttatccagcttggattgcttatccaacatagtggaccggatcacctcctcagagcaacctgggttgcctctccaggatctggcttctctctctccagttgccagcaccgattcacagcctgcaactccaggggcttctagttccaggcttatctatcatgtgctatgaactaattttctggtctatatgacttcttccaggagggcctaatacacaggaagaagaaggcttcaaaaagtcccaaaccaagacaacatgtacataaagatttcttttcagttgtaaatttgtaaagattaccttgccactttataagaaagtgtatttaactaaaaagtcatcattgcaaataatactttcttcttctttattattctttgcttagatattaatacatagttccagtaatactatttctgatagggggccattgattgagggtagcttgttgcaatgcttaacttatatatacatatatatatattataaatattgctcatcaaaatgtctctggtgtttagagctttatttttttctttaaaacattaaaacagctgagaatcagttaaatggaattttaaatatatttaactatttcttttctctttaatcctttagttatattgtattaaataaaaatataatactgcctaatgtatatattttgatcttttcttgtaagaaatgtatcttttaaatgtaagcacaaaatagtactttgtggatcatttcaagatataagaaattttggaaattccaccataaataaaattttttactacaag

SEQ ID NO: 45 - protein, 255 aa, Homo sapiens
MDVMDGCQFSPSEYFYDGSCIPSPEGEFGDEFVPRVAAFGAHKAELQGSDEDEHVRAPTGHHQAGHCLMWACKACKRKSTTMDRRKAATMRERRRLKKVNQAFETLKRCTTTNPNQRLPKVEILRNAIRYIESLQELLREQVENYYSLPGQSCSEPTSPTSNCSDGMPECNSPVWSRKSSTFDSIYCPDVSNVYATDKNSLSSLDCLSNIVDRITSSEQPGLPLQDLASLSPVASTDSQPATPGASSSRLIYHVL

SEQ ID NO: 46 - DNA, 1912 bp, Homo sapiens
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 47 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 48 - DNA, 2615 bp, Homo sapiens
actcgccgcagcctgcgcgccttctccagtccgcggtgccatggcccccgcccgtctgttcgcgctgctgctgttcttcgtaggcggagtcgccgagtcgatccgagagactgaggtcatcgacccccaggacctcctagaaggccgatacttctccggagccctaccagacgatgaggatgtagtggggcccgggcaggaatctgatgactttgagctgtctggctctggagatctggatgacttggaagactccatgatcggccctgaagttgtccatcccttggtgcctctagataaccatatccctgagagggcagggtctgggagccaagtccccaccgaacccaagaaactagaggagaatgaggttatccccaagagaatctcacccgttgaagagagtgaggatgtgtccaacaaggtgtcaatgtccagcactgtgcagggcagcaacatctttgagagaacggaggtcctggcagctctgattgtgggtggcatcgtgggcatcctctttgccgtcttcctgatcctactgctcatgtaccgtatgaagaagaaggatgaaggcagctatgacctgggcaagaaacccatctacaagaaagcccccaccaatgagttctacgcgtgaagcttgcttgtgggcactggcttggactttagcggggagggaagccaggggattttgaagggtggacattagggtagggtgaggtcaacctaatactgacttgtcagtatctccagctctgattacctttgaagtgttcagaagagacattgtcttctactgttctgccaggttcttcttgagctttgggcctcagttgccctggcagaaaaatggattcaacttggcctttctgaaggcaagactgggattggatcacttcttaaacttccagttaagaatctaggtccgccctcaagcccatactgaccatgcctcatccagagctcctctgaagccagggggctaacggatgttgtgtggagtcctggctggaggtcctcccccagtggccttcctcccttcctttcacagccggtctctctgccaggaaatgggggaaggaactagaaccacctgcaccttgagatgtttctgtaaatgggtacttgtgatcacactacgggaatctctgtggtatatacctggggccattctaggctctttcaagtgacttttggaaatcaaccttttttatttgggggggaggatggggaaaagagctgagagtttatgctgaaatggatttatagaatatttgtaaatctatttttagtgtttgttcgtttttttaactgttcattcctttgtgcagagtgtatatctctgcctgggcaagagtgtggaggtgccgaggtgtcttcattctctcgcacatttccacagcacctgctaagtttgtatttaatggtttttgtttttgtttttgtttgtttcttgaaaatgagagaagagccggagagatgatttttattaattttttttttttttttttttttttactatttatagctttagatagggcctcccttcccctcttctttctttgttctctttcattaaaccccttccccagttttttttttatactttaaaccccgctcctcatggccttggccctttctgaagctgcttcctcttataaaatagcttttgccgaaacatagtttttttttagcagatcccaaaatataatgaaggggatggtgggatatttgtgtctgtgttcttataatatattattattcttccttggttctagaaaaatagataaatatatttttttcaggaaatagtgtggtgtttccagtttgatgttgctgggtggttgagtgagtgaattttcatgtggctgggtgggtttttgcctttttctcttgccctgttcctggtgccttctgatggggctggaatagttgaggtggatggttctaccctttctgccttctgtttgggacccagctggtgttctttggtttgctttcttcaggctctagggctgtgctatccaatacagtaaccacatgcggctgtttaaagttaagccaattaaaatcacataagattaaaaattccttcctcagttgcactaaccacgtttctagaggcgtcactgtatgtagttcatggctactgtactgacagcgagagcatgtccatctgttggacagcactattctagagaactaaactggcttaacgagtcacagcctcagctgtgctgggacgacccttgtctccctgggtaggggggggggaatgggggagggctgatgaggccccagctggggcctgttgtctgggaccctccctctcctgagaggggaggcctggtggcttagcctgggcaggtcgtgtctcctcctgaccccagtggctgcggtgaggggaaccaccctcccttgctgcaccagtggccattagctcccgtcaccactgcaacccagggtcccagctggctgggtcctcttctgcccccagtgcccttccccttgggctgtgttggagtgagcacctcctctgtaggcacctctcacactgttgtctgttactgattttttttgataaaaagataataaaacctggtactttctaaaaa

SEQ ID NO: 49 - protein, 198 aa, Homo sapiens
MAPARLFALLLFFVGGVAESIRETEVIDPQDLLEGRYFSGALPDDEDVVGPGQESDDFELSGSGDLDDLEDSMIGPEVVHPLVPLDNHIPERAGSGSQVPTEPKKLEENEVIPKRISPVEESEDVSNKVSMSSTVQGSNIFERTEVLAALIVGGIVGILFAVFLILLLMYRMKKKDEGSYDLGKKPIYKKAPTNEFYA

SEQ ID NO: 50 - DNA, 1576 bp, Homo sapiens
aaatggcacccagcagttggcgtgaggggctgctggagcttgggggctggtggcaggaacaagccttttccgaccccatggagctgtatgagacatccccctacttctaccaggaaccccgcttctatgatggggaaaactacctgcctgtccacctccagggcttcgaaccaccaggctacgagcggacggagctcaccctgagccccgaggccccagggccccttgaggacaaggggctggggacccccgagcactgtccaggccagtgcctgccgtgggcgtgtaaggtgtgtaagaggaagtcggtgtccgtggaccggcggcgggcggccacactgagggagaagcgcaggctcaagaaggtgaatgaggccttcgaggccctgaagagaagcaccctgctcaaccccaaccagcggctgcccaaggtggagatcctgcgcagtgccatccagtacatcgagcgcctccaggccctgctcagctccctcaaccaggaggagcgtgacctccgctaccggggcgggggcgggccccagccaggggtgcccagcgaatgcagctctcacagcgcctcctgcagtccagagtggggcagtgcactggagttcagcgccaacccaggggatcatctgctcacggctgaccctacagatgcccacaacctgcactccctcacctccatcgtggacagcatcacagtggaagatgtgtctgtggccttcccagatgaaaccatgcccaactgagattgtcttccaagccgggcatccttgcgagccccccaagctggccacagatgccactacttctgtagcaggggcctcctaagccaggctgccctgatgctaggaagccagctctggggtgccataggccagactatccccttcctcatccatgtaaggttaacccaccccccagcaagggactggacgccctcattcagctgcctccttagaggagagggcatcccctttccagggaggtaaagcaggggaccagagcgccccctcgtgtatgccccagctcagggggcaaactcaggagcttcctttttatcataacgcggcctctaattccaccccccaagtgaaacggtttgagagacgcagtgccctgacctggacaagctgtgcacgtctcctgttctggtctcttcccgatgccagtggctgggctgggcctgccctgaattgagagagaagaaggggagaggaacagccctctgttcccaagtccctggggggccaaacttttgcagtgaatattgggaaccttccagtggttttatgttttgttttgtttcgtgtgttgtttgtaaagctgccatccgaccaaggtctcctgtgctgaagttgccggggacaggcagggaaaaggggttggggcctcttgggggtgatttcttttgttaacaaagcattgtgtggttttgccattgttttgtattttttttttttttttttttttttgctaacttatttggatttccttttttaaaaaatgaataaagactggttgccagaaaaaaaaaaaaaaaaaaaaaaaaaaaaaaaaaaaaaaaaaaaaa

SEQ ID NO: 51 - protein, 224 aa, Homo sapiens
MELYETSPYFYQEPRFYDGENYLPVHLQGFEPPGYERTELTLSPEAPGPLEDKGLGTPEHCPGQCLPWACKVCKRKSVSVDRRRAATLREKRRLKKVNEAFEALKRSTLLNPNQRLPKVEILRSAIQYIERLQALLSSLNQEERDLRYRGGGGPQPGVPSECSSHSASCSPEWGSALEFSANPGDHLLTADPTDAHNLHSLTSIVDSITVEDVSVAFPDETMPN

SEQ ID NO: 52 - DNA, 1823 bp, Homo sapiens
gagaagctaggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggatatggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctgaggggatggtggccgcccacccgcccgagggatggtgcccctagggtccctcgcgcccaaaagattgaacttaaatgcccccctcccaacagcgctttaaaagcgacctctcttgaggtaggagaggcgggagaactgaagtttccgcccccgccccacagggcaaggacacagcgcggttttttccacgcagcacccttctcggagacccattgcgatggccgctccgtgttcctcggtgggccagagctgaaccttgaggggctaggttcagctttctcgcgccctcccccatgggggtgagaccctcgcagacctaagccctgccccgggatgcaccggttatttgggggggcgtgagacccagtgcactccggtcccaaatgtagcaggtgtaaccgtaacccacccccaacccgtttcccggttcaggaccactttttgtaatacttttgtaatctattcctgtaaataagagttgctttgccagagcaggagcccctggggctgtatttatctctgaggcatggtgtgtggtgctacagggaatttgtacgtttataccgcaggcgggcgagccgcgggcgctcgctcaggtgatcaaaataaaggcgctaatttataaaaaaaaaaaaaaaaaa

SEQ ID NO: 53 - protein, 320 aa, Homo sapiens
MELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYEGAYYNEAPSEPRPGKSAAVSSLDCLSSIVERISTESPAAPALLLADVPSESPPRRQEAAAPSEGESSGDPTQSPDAAPQCPAGANPNPIYQVL

SEQ ID NO: 54 - DNA, 20 bp, Artificial Sequence
  Primer sequence
tcggctctaggttccatgtc

SEQ ID NO: 55 - DNA, 20 bp, Artificial Sequence
  Primer sequence
agcagatccctgcttctcaa

SEQ ID NO: 56 - DNA, 21 bp, Artificial Sequence
  Primer sequence
gatcacttgaaaatgctccac

SEQ ID NO: 57 - DNA, 20 bp, Artificial Sequence
  Primer sequence
tacctgatgacctcggtgag

SEQ ID NO: 58 - DNA, 20 bp, Artificial Sequence
  Primer sequence
agccgcaaaactaccaagaa

SEQ ID NO: 59 - DNA, 20 bp, Artificial Sequence
  Primer sequence
cgtgagacgctccatacaga

SEQ ID NO: 60 - DNA, 20 bp, Artificial Sequence
  Primer sequence
aagattctctgccgctacca

SEQ ID NO: 61 - DNA, 20 bp, Artificial Sequence
  Primer sequence
tcacagtgtccgtccttcag

SEQ ID NO: 62 - DNA, 20 bp, Artificial Sequence
  Primer sequence
ggcctgcctgaatgtaacag

SEQ ID NO: 63 - DNA, 20 bp, Artificial Sequence
  Primer sequence
gttgctcggagttggtgatt

SEQ ID NO: 64 - DNA, 20 bp, Artificial Sequence
  Primer sequence
ggctggcatccagttacatc

SEQ ID NO: 65 - DNA, 20 bp, Artificial Sequence
  Primer sequence
gcggaaacttctttggtgtc

SEQ ID NO: 66 - DNA, 20 bp, Artificial Sequence
  Primer sequence
cgattcgctacatcgaaggt

SEQ ID NO: 67 - DNA, 20 bp, Artificial Sequence
  Primer sequence
aggtgccatcgtagcagttc

SEQ ID NO: 68 - DNA, 25 bp, Artificial Sequence
  Primer sequence
gagctccatatatacccttcagata

SEQ ID NO: 69 - DNA, 20 bp, Artificial Sequence
  Primer sequence
ggtaacccatgaccaggatg

SEQ ID NO: 70 - DNA, 20 bp, Artificial Sequence
  Primer sequence
tgacccaagtcctgtgtgag

SEQ ID NO: 71 - DNA, 20 bp, Artificial Sequence
  Primer sequence
gtcttgcgggaatagctctg

SEQ ID NO: 72 - DNA, 24 bp, Artificial Sequence
  Primer sequence
gaaccagttcttcgtgatagagga

SEQ ID NO: 73 - DNA, 20 bp, Artificial Sequence
  Primer sequence
tgtcttggtggcatgaatgt

SEQ ID NO: 74 - DNA, 19 bp, Artificial Sequence
  Primer sequence
atcaagggcttccgcaatg

SEQ ID NO: 75 - DNA, 22 bp, Artificial Sequence
  Primer sequence
cacagtcgatggtggagaacag

(19) United States
(12) Patent Application Publication
(10) Pub. No.: US 2015/0166961 A1
(43) Pub. Date: Jun. 18, 2015

(54) EX VIVO EXPANSION OF MYOGENIC STEM CELLS BY NOTCH ACTIVATION

(71) Applicant: Fred Hutchinson Cancer Research Center, Seattle, WA (US)

(72) Inventors: Maura H. Parker, Lynnwood, WA (US); Stephen J. Tapscott, Seattle, WA (US)

(21) Appl. No.: 14/407,446

(22) PCT Filed: Jun. 14, 2013
(86) PCT No.: PCT/US13/46028

§ 371 Date: Dec. 11, 2014

Related U.S. Application Data
(60) Provisional application No. 61/659,912, filed on Jun. 14, 2012.

(51) Int. Cl. C12N 5/077; A61K 35/34

(52) U.S. Cl. CPC C12N 5/0658; A61K 35/34; C12N 2501/415; C12N 2501/42

(57) ABSTRACT

Activating Notch signaling in cultured canine muscle derived cells inhibited myogenic differentiation, and increased the number of myogenic progenitor cells that were similar to quiescent or newly activated satellite cells. Importantly, cells expanded in the presence of Notch activation maintained engraftment potential, indicating the potential for therapeutic benefit. Activation of Notch signaling to inhibit myogenic differentiation in cultured human muscle-derived cells is also contemplated, for maintaining engraftment potential using such human cells in transplantation.

### CROSS REFERENCE TO RELATED APPLICATION

This application claims the benefit of U.S. provisional patent application Ser. No. 61/659,912, filed Jun. 14, 2012, which is incorporated herein by reference in its entirety.

### STATEMENT OF GOVERNMENT INTEREST

This invention was made with government support under Grant No. P01-NS046788-07 awarded by the National Institute of Neurological Disorders and Stroke, and Grant No. U01-HL100395 awarded by the National Heart, Lung, and Blood Institute. The government has certain rights in this invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_{—}415WO_SEQUENCE_LISTING_.txt. The text file is 172 KB, was created on Jun. 14, 2013, and is being submitted electronically via EFS-Web.

### BACKGROUND

1. Technical Field

The present disclosure relates generally to tissue repair by stem cell transplantation. More specifically, compositions and methods are described herein that relate to repair of muscle tissue such as dystrophic muscle by transplantation of myogenic stem cells that are propagated ex vivo in a manner that preserves their engraftment potential.

2. Description of the Related Art

Duchenne Muscular Dystrophy (DMD), the most common and severe form of muscular dystrophy, is caused by mutations in the dystrophin gene, the largest gene identified in the human genome. Transplantation of myogenic stem cells possesses great potential for long-term repair of dystrophic muscle. Indeed, intramuscular injection of adult satellite cell-derived myoblasts from a normal syngeneic donor into mdx mice results in the formation of dystrophin-positive muscle fibers [1, 2, 3]. Furthermore, intramuscular injection of allogeneic donor muscle-derived cells into chimeric cxmd canine recipients restored dystrophin expression for at least 24 weeks in the absence of post-transplant immunosuppression, indicating that cell transplantation may be a viable therapeutic option for muscular dystrophy [4].

The ability of single muscle fibers to engraft more effectively than mononuclear cell preparations suggests that association of the satellite cell with the fiber preserves the ability of the satellite cell to participate in muscle repair. In mouse studies, physical trituration of the fibers to disrupt satellite cell-fiber interactions yields cells with significantly greater engraftment potential than cells enzymatically removed from the fiber [6]. The authors of [6] hypothesize that enzymatic disruption may cleave cell surface proteins required for donor cell engraftment. However, it is also possible that time away from the fiber or niche has a negative effect on donor satellite cell engraftment. Indeed, culturing muscle-derived cells on a substrate with a similar stiffness to normal skeletal muscle (12 kPa) improves donor cell engraftment, indicating that biophysical signaling is important for satellite cell stemness [18, 19].

Activation of Notch signaling is important for satellite cell proliferation and muscle regeneration after injury [13]. New evidence indicates that Notch activity also plays a significant role in maintenance of the satellite cell population after injury, and that expression of Notch target genes is associated with quiescent satellite cells that express high levels of Pax7 [20, 21].

However, multiple muscle groups within the body will need to be targeted, and a single donor muscle biopsy is unlikely to provide enough cells to effectively transplant the muscle mass of a patient affected by muscular dystrophy. Traditional means of expanding satellite cell-derived myoblasts ex vivo results in a dramatic loss of engraftment potential [4, 5]. The success of single muscle fiber transplantation suggests that mimicking the biochemical and biophysical signaling from the fiber may be important for maintaining engraftment potential of expanded muscle satellite cells [6, 7].

Expansion of hematopoietic progenitor cells on Notch ligand maintains their engraftment potential [8-12]. Skeletal muscle injury in mice results in increased expression of Delta-like-1(DII-1) within the niche, and activation of Notch signaling increases the number of proliferating myogenic cells and promotes muscle regeneration after injury [13]. In vitro, overexpression of an activated form of Notch downregulates expression of MyoD and myogenin and inhibits myogenic differentiation in primary mouse myoblasts and C2C12 cells [13, 14]. The extracellular domain of DII-1 fused to the Fc portion of human IgG (Delta-1^{ext}-IgG) is sufficient for inhibition of differentiation in cultured C2C12 myoblasts; however, immobilization is required for effective signaling [15].

Clearly there remains a need for improved compositions and methods for obtaining increased numbers of myogenic stem cells for use in transplantation such as for muscle tissue repair, including compositions and methods for expanding such cells ex vivo while maintaining their potential for engraftment in vivo. The presently described embodiments address these needs and provide other related advantages.

### BRIEF SUMMARY

According to certain embodiments of the present invention, there is provided an ex vivo method for expanding myogenic precursor cells while preserving engraftment potential in one or more of said myogenic precursor cells, the method comprising activating Notch signaling in one or a plurality of myogenic precursor cells that are present in a population of cells isolated from skeletal muscle, said step of activating taking place in vitro under conditions and for a time sufficient for expansion of the myogenic precursor cells in the population of cells to obtain one or a plurality of myogenic precursor cells in which Notch signaling is detectably activated in a statistically significant manner to a greater degree than in control cells that do not undergo said step of activating, and thereby expanding the myogenic precursor cells while preserving engraftment potential in one or more of said cells.

In certain further embodiments the step of activating Notch signaling comprises contacting the population of cells with an immobilized Notch ligand. In certain still further embodiments the Notch ligand comprises a polypeptide selected from a eukaryotic Notch ligand delta family member and a eukaryotic Notch ligand serrate family member. In certain embodiments the eukaryotic Notch ligand delta family member is selected from human delta-like-1 (DLL1, UniProt ID O00548 (SEQ ID NO: 1), Genbank ACH57449 (SEQ ID NO: 2), Genbank NP_{—}005609.3 (SEQ ID NO: 3)), delta-like-3 (DLL3, cDNA (var. 1)—NM_{—}016941 (SEQ ID NO; 4); protein (var. 1)—NP_{—}058637.1 (SEQ ID NO: 5); cDNA (var. 2)—NM_{—}203486 (SEQ ID NO: 6); protein (var. 2)—NP_{—}982353.1 (SEQ ID NO: 7)), delta-like-4 (DLL4, cDNA—NM_{—}019074 (SEQ ID NO: 8); protein—NP_{—}061947.1 (SEQ ID NO: 9)), Dlk1 (NP_{—}003827.3 (SEQ ID NO: 10); cDNA—NM_{—}003836 (SEQ ID NO: 11)), Dlk2 (NP_{—}076421.2 (SEQ ID NO: 12) (var. 1), NP_{—}996262.1 (SEQ ID NO: 13) (var. 2); cDNA—NM_{—}023932 (SEQ ID NO: 14) (var. 1) and NM_{—}206539 (SEQ ID NO: 15) (var. 2)), MAGP1/MFAP2 (NP_{—}059453.1 (SEQ ID NO: 16) (var. 1), NP_{—}002394.1 (SEQ ID NO: 17) (var. 2), NP_{—}001128719.1 (SEQ ID NO: 18) (var. 3), NP_{—}001128720.1 (SEQ ID NO: 19) (var. 4); cDNA—NM_{—}017459 (SEQ ID NO: 20) (var. 1), NM_{—}002403 (SEQ ID NO: 21) (var. 2), NM_{—}001135247 (SEQ ID NO: 22) (var. 3), NM_{—}001135248 (SEQ ID NO: 23) (var. 4)), MAGP2/MFAP5 (NP_{—}003471.1 (SEQ ID NO: 24); cDNA—NM_{—}003480 (SEQ ID NO: 25)), JAG1 (NM_{—}000214 (SEQ ID NO: 26); protein—NP_{—}000205.1 (SEQ ID NO: 27)) and JAG2 (NM_{—}002226 (SEQ ID NO: 28); protein—NP_{—}002217.3 (SEQ ID NO: 29)).

In certain embodiments the Notch ligand comprises an extracellular domain of human delta-like-1 (DLL1, UniProt ID O00548 (SEQ ID NO: 1), Genbank ACH57449 (SEQ ID NO:2), Genbank NP_{—}005609.3 (SEQ ID NO: 3)) or a polypeptide that has at least 80% sequence identity to said extracellular domain and is capable of activating Notch signaling. In certain other embodiments the immobilized Notch ligand comprises a fusion protein which comprises a Notch ligand polypeptide fused to a fusion domain polypeptide. In certain further embodiments the fusion domain polypeptide is selected from an immunoglobulin constant region polypeptide, a GST polypeptide, a streptavidin polypeptide, a maltose binding protein polypeptide, a c-myc polypeptide, a yeast Aga2p polypeptide, a filamentous phage coat protein polypeptide, a FLAG polypeptide, and a calmodulin binding peptide (CBP). According to certain other embodiments the immobilized Notch ligand is expressed on cell surfaces of a feeder cell layer that is present during said step of contacting.

According to certain embodiments, detectably activated Notch signaling comprises a statistically significant increase in expression by the myogenic precursor cells of at least one marker gene selected from the group consisting of Hey1 (NM_{—}001002953 (SEQ ID NO: 30) (canine cDNA); NP_{—}001002953.1 (SEQ ID NO: 31) (canine protein); NM_{—}012258 (SEQ ID NO: 32) (human var. 1 cDNA); NP_{—}036390.3 (SEQ ID NO: 33) (human var. 1 protein); NM_{—}001040708 (SEQ ID NO: 34) (human var. 2 cDNA); NP_{—}001035798.1 (SEQ ID NO: 35) (human var. 2 protein), HeyL (NM_{—}014571 (SEQ ID NO: 36) (human cDNA); NP_{—}055386.1 (SEQ ID NO: 37) (human protein)) and Dtx4 (NM_{—}015177 (SEQ ID NO: 38) (human cDNA); NP_{—}055992.1 (SEQ ID NO: 39) (human protein)), relative to expression of the marker gene by myogenic precursor cells that do not undergo the step of activating Notch signaling.

According to certain other embodiments, detectably activated Notch signaling comprises inhibition of differentiation of the myogenic precursor cells that manifests as one or more of (i) a statistically significant increase in expression by the myogenic precursor cells of at least one marker gene selected from the group consisting of Pax7 (NM_{—}002584 (SEQ ID NO: 40) (human cDNA); NP_{—}002575.1 (SEQ ID NO: 41) (human protein)), musculin (NM_{—}005098 (SEQ ID NO: 42) (human cDNA); NP_{—}005089.2 (SEQ ID NO: 43) (human protein)), Myf5 (NM_{—}005593 (SEQ ID NO: 44) (human cDNA); NP_{—}005584.2 (SEQ ID NO: 45) (human protein)), CXCR4 (NM_{—}001008540 (SEQ ID NO: 46) (human cDNA); NP_{—}001008540.1 (SEQ ID NO: 47) (human protein)) and syndecan4 (NM_{—}002999 (SEQ ID NO: 48) (human cDNA); NP_{—}002990.2 (SEQ ID NO: 49) (human protein)), relative to expression of the marker gene by myogenic precursor cells that do not undergo the step of activating Notch signaling, and (ii) a statistically significant decrease in expression by the myogenic precursor cells of at least one marker gene selected from the group consisting of myogenin (NM_{—}002479 (SEQ ID NO: 50) (human cDNA); NP_{—}002470.2 (SEQ ID NO: 51) (human protein)) and MyoD (NM_{—}002478 (SEQ ID NO: 52) (human cDNA); NP_{—}002469.2 (SEQ ID NO: 53) (human protein)), relative to expression of the marker gene by myogenic precursor cells that do not undergo the step of activating Notch signaling.

In certain embodiments any of the above described methods further comprises contacting a Wnt ligand, or a Wnt ligand receptor agonist, with the one or plurality of myogenic precursor cells in which Notch signaling is activated. In certain still further embodiments at least one of: (a) the Wnt ligand is Dkk2; (b) the Wnt ligand receptor agonist is capable of signaling via Fzd4; (c) the Wnt ligand is selected from human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, Dkk-1, Dkk-2, Dkk-4, sFRP-1, sFRP-2, sFRP-3, sFRP4, sFRP-5, WIF-1, Norrin, R-spondin, and DkkL1; and (d) the Wnt ligand receptor agonist is capable of activating a canonical or non-canonical Wnt signaling pathway via at least one of FZD1, FZD2, FZD3, FZD4, FZD5, FZD6, FZD7, FZD8, FZD9, LRP5, LRP6, ROR1, ROR2, RYK, MuSK, and a glypican.

Turning to another embodiment, there is provided an ex vivo method for expanding myogenic precursor cells while preserving engraftment potential in one or more of said myogenic precursor cells, the method comprising activating Notch signaling in one or a plurality of myogenic precursor cells that are present in a population of cells isolated from skeletal muscle by contacting the population of cells with an immobilized Notch ligand, said step of activating taking place in vitro under conditions and for a time sufficient for expansion of the myogenic precursor cells in the population to obtain one or more myogenic precursor cells in which Notch signaling is detectably activated in a statistically significant manner to a greater degree than in control cells that do not undergo said step of activating, and thereby expanding the myogenic precursor cells while preserving engraftment potential in one or more of said cells. In a further embodiment, the immobilized Notch ligand comprises a fusion protein which comprises (i) an extracellular domain of human delta-like-1 (DLL1, UniProt ID O00548 (SEQ ID NO: 1), Genbank ACH57449 (SEQ ID NO: 2), Genbank NP_{—}005609.3 (SEQ ID NO: 3)) or a polypeptide that has at least 80%, 85%, 86%, 87%, 88%, 89%, 90%, 91%, 92%, 93%, 94%, or 95% sequence identity to said extracellular domain and is capable of activating Notch signaling, fused to (ii) an immunoglobulin constant region polypeptide. In certain further embodiments the method further comprises contacting a Wnt ligand, or a Wnt ligand receptor agonist, with the one or plurality of myogenic precursor cells in which Notch signaling is activated. In certain still further embodiments at least one of: (a) the Wnt ligand is Dkk2; (b) the Wnt ligand receptor agonist is capable of signaling via Fzd4; (c) the Wnt ligand is selected from human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, Dkk-1, Dkk-2, Dkk-4, sFRP-1, sFRP-2, sFRP-3, sFRP4, sFRP-5, WIF-1, Norrin, R-spondin, and DkkL1; and (d) the Wnt ligand receptor agonist is capable of activating a canonical or non-canonical Wnt signaling pathway via at least one of FZD1, FZD2, FZD3, FZD4, FZD5, FZD6, FZD7, FZD8, FZD9, LRP5, LRP6, ROR1, ROR2, RYK, MuSK, and a glypican.

In another embodiment the present invention provides a composition comprising ex vivo expanded myogenic precursor cells in which engraftment potential is preserved, said composition being formed by a method which comprises activating Notch signaling in one or a plurality of myogenic precursor cells that are present in a population of cells isolated from skeletal muscle by contacting the population of cells with an immobilized Notch ligand, said step of activating taking place in vitro under conditions and for a time sufficient for expansion of the myogenic precursor cells in the population of cells to obtain one or a plurality of myogenic precursor cells in which Notch signaling is detectably activated in a statistically significant manner to a greater degree than in control cells that do not undergo said step of activating, and thereby expanding the myogenic precursor cells while preserving engraftment potential in one or more of said cells. In certain further embodiments the method by which the composition is formed further comprises contacting a Wnt ligand, or a Wnt ligand receptor agonist, with the one or plurality of myogenic precursor cells in which Notch signaling is activated. In certain still further embodiments at least one of: (a) the Wnt ligand is Dkk2; (b) the Wnt ligand receptor agonist is capable of signaling via Fzd4; (c) the Wnt ligand is selected from human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, Dkk-1, Dkk-2, Dkk-4, sFRP-1, sFRP-2, sFRP-3, sFRP4, sFRP-5, WIF-1, Norrin, R-spondin, and DkkL1; and (d) the Wnt ligand receptor agonist is capable of activating a canonical or non-canonical Wnt signaling pathway via at least one of FZD1, FZD2, FZD3, FZD4, FZD5, FZD6, FZD7, FZD8, FZD9, LRP5, LRP6, ROR1, ROR2, RYK, MuSK, and a glypican.

In another embodiment there is provided a method for promoting muscle tissue regeneration in a mammal, comprising: (a) activating Notch signaling, in one or a plurality of myogenic precursor cells that are present in a population of cells isolated from skeletal muscle, by contacting the population of cells with an immobilized Notch ligand, said step of activating taking place in vitro under conditions and for a time sufficient for expansion of the myogenic precursor cells in the population of cells to obtain one or a plurality of myogenic precursor cells in which Notch signaling is detectably activated, in a statistically significant manner to a greater degree than in control cells that do not undergo said step of activating, and thereby obtaining myogenic precursor cells having increased engraftment potential in a statistically significant manner relative to control cells that do not undergo said step of activating; and (b) administering said myogenic precursor cells that have increased engraftment potential to a transplantation site in a mammal, and thereby promoting muscle regeneration. In certain further embodiments, the immobilized Notch ligand comprises a fusion protein which comprises (i) an extracellular domain of human delta-like-1 (DLL1, UniProt ID O00548 (SEQ ID NO: 1), Genbank ACH57449 (SEQ ID NO: 2), Genbank NP_{—}005609.3 (SEQ ID NO: 3)) or a polypeptide that has at least 80%, 85%, 86%, 87%, 88%, 89%, 90%, 91%, 92%, 93%, 94%, or 95% sequence identity to said extracellular domain and is capable of activating Notch signaling, fused to (ii) an immunoglobulin constant region polypeptide.

In certain further embodiments the method further comprises contacting a Wnt ligand, or a Wnt ligand receptor agonist, with the one or plurality of myogenic precursor cells in which Notch signaling is activated. In certain still further embodiments at least one of: (a) the Wnt ligand is Dkk2; (b) the Wnt ligand receptor agonist is capable of signaling via Fzd4; (c) the Wnt ligand is selected from human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, Dkk-1, Dkk-2, Dkk-4, sFRP-1, sFRP-2, sFRP-3, sFRP4, sFRP-5, WIF-1, Norrin, R-spondin, and DkkL1; and (d) the Wnt ligand receptor agonist is capable of activating a canonical or non-canonical Wnt signaling pathway via at least one of FZD1, FZD2, FZD3, FZD4, FZD5, FZD6, FZD7, FZD8, FZD9, LRP5, LRP6, ROR1, ROR2, RYK, MuSK, and a glypican.

These and other aspects of the herein described invention embodiments will be evident upon reference to the following detailed description and attached drawings. All of the U.S. patents, U.S. patent application publications, U.S. patent applications, foreign patents, foreign patent applications and non-patent publications referred to in this specification and/or listed in the Application Data Sheet are incorporated herein by reference in their entirety, as if each was incorporated individually. Aspects and embodiments of the invention can be modified, if necessary, to employ concepts of the various patents, applications and publications to provide yet further embodiments.

### BRIEF DESCRIPTION OF THE SEVERAL VIEWS OF THE DRAWINGS

FIG. 1. Cultured myoblasts displayed poor engraftment. Cryosections from NOD/SCID mouse muscle injected with 5×10⁴ fresh canine muscle-derived cells or 5 single muscle fibers were immunostained with anti-dystrophin, or anti-Pax7 and anti-lamin A/C, and fluorescently labeled secondary antibodies. The number of fibers expressing canine dystrophin (A) and the number of nuclei expressing Pax7 and canine lamin A/C (B) per cross-section were counted using cryosections surrounding the region of highest engraftment within the muscle. For the fiber transplants in (A), the bars represent the average±SD (n≧3 cryosections per mouse). For all others, the bars represent the average of the averages (n≧3 cryosections per mouse, n=3 mice per cell dose). (C) Cryosections from NOD/SCID mouse muscle injected with 1×10⁴ or 5×10⁴ fresh canine muscle-derived cells or cells expanded ex vivo for 8 days were immunostained with anti-dystrophin and fluorescently labeled secondary antibodies. The bars represent the average of the averages±SD (n≧3 cryosections per mouse, n=3 mice per cell dose). A Student's t-test was used to determine statistical significance (* p<0.05; ** p<0.01).

FIG. 2. Delta-1^{ext}-IgG inhibited canine muscle cell differentiation. (A) Established canine satellite cell-derived myoblasts, or (B) freshly isolated canine muscle-derived cells were cultured on plates coated with Delta-1^{ext}-IgG or human IgG. After 8 days, the cells were fixed and immunostained with anti-Pax7 (green), and anti-myogenin (red).

FIG. 3 (A-C). Notch activation altered gene expression in canine muscle-derived cells. cDNA was generated from RNA isolated from canine muscle-derived cells cultured on plates coated with Delta-1^{ext}-IgG (darker bars) or human IgG (lighter bars), and used for quantitative PCR using the primers indicated. The bars represent the average expression level relative to TIMM17B±SD (n=3). A Student's t-test was used to determine statistical significance (* p<0.05; ** p<0.01).

FIG. 4. Expanded canine muscle-derived cells expressed a higher level of CXCR4. Freshly isolated canine muscle-derived cells, cultured on plates coated with Delta-1^{ext}-IgG or human IgG, were incubated with anti-CXCR4 (A) or anti-syndecan 4 (B), and Alexa Fluor 488-labeled secondary antibody, or isotype control and AlexaFluor 488-labeled secondary antibody, and sorted using FACS. The resulting histograms are vertically offset, and scaled to avoid overlap.

FIG. 5. Cells expanded on Delta-1^{ext}-IgG maintained engraftment. Cryosections from mouse muscle injected with 1×10⁴ or 5×10⁴ freshly isolated mixed canine muscle-derived mononuclear cells, cells expanded on Delta-1^{ext}-IgG, or cells expanded on human IgG, were immunostained with anti-dystrophin, anti-lamin A/C, and/or anti-Pax7 antibodies and fluorescently labeled secondary antibody. The number of fibers expressing canine dystrophin (A), the number of nuclei expressing canine lamin A/C (B), the number of nuclei expressing canine lamin A/C and Pax7 (C) and the ratio of the number of nuclei expressing canine lamin A/C to the number of fibers expressing canine dystrophin per cross-section (E) were determined. The bars represent the average of the averages±SD (n≧3 cryosections per mouse, n=3 mice per cell dose). (D) Cryosections from mouse muscle injected with 1×10⁴ freshly isolated cells expanded on Delta-1^{ext}-IgG, established canine myoblasts expanded on Delta-1^{ext}-IgG, or established canine myoblasts expanded on human IgG, were immunostained with anti-dystrophin. The number of fibers expressing canine dystrophin per cross-section was determined. The bars represent the average of the averages±SD (n≧3 cryosections per mouse, n=3 mice per cell dose). For all data, a Student's t-test was used to determine statistical significance (* p<0.05; ** p<0.01).

FIG. 6. Delta-1^{ext}-IgG expanded cells functioned as long-term repopulating cells. (A) Two groups of mice were injected with 1×10⁴ freshly isolated canine muscle-derived cells or cells expanded on Delta-1^{ext}-IgG. The transplanted muscle of group 1 was harvested 12 weeks after cell injection. The transplanted muscle of group 2 was injected with 1.2% BaCl₂ 4 and 8 weeks after cell injection, and harvested 12 weeks after cell injection. (B,C) Cryosections from the experiment outlined in (A) were immunostained with anti-dystrophin, or anti-lamin A/C and anti-Pax7 antibodies, and fluorescently labeled secondary antibodies. The number of fibers expressing canine dystrophin (B), and the number of nuclei expressing canine lamin A/C and Pax7 (C) per cross-section was determined. The bars represent the average of the averages±SD (n≧3 cryosections per mouse, n=3 mice per cell dose). (D) Cryosections were immunostained with anti-dystrophin (red) and anti-developmental myosin heavy chain (green), and fluorescently labeled secondary antibodies. (E) The fraction of canine dystrophin-positive fibers expressing developmental myosin heavy chain (devMyHC) was determined. The bars represent the average of the averages±SD (n≧3 cryosections per mouse, n=3 mice per cell dose). (F-I) Two groups of 3 mice were injected with 5×10⁴ freshly isolated canine muscle-derived cells or cells expanded on Delta-1^{ext}-IgG. The mixed population of muscle-derived cells was isolated from each injected muscle and transplanted into a secondary recipient. Muscle from the secondary recipients was harvested 4 weeks after injection, and cryosections immunostained with anti-dystrophin (F), or anti-lamin A/C and anti-Pax7 antibodies (H), and fluorescently labeled secondary antibodies. The number of fibers expressing canine dystrophin (G), and the number of nuclei expressing canine lamin A/C and Pax7 (I) per cross-section was determined. The bars represent the average±SD from each mouse (n=3 cryosections per mouse).

FIG. 7. Notch activation upregulates components of Wnt signaling pathway. (A) RNA was isolated from proliferating myoblasts (lanes 1, 2), from cells expanded on Delta-1^{ext}-IgG (lane 3), and from cells expanded on human IgG (lane 4). RT-PCR was performed using the primers indicated. (B) RT-quantitative PCR was performed using RNA isolated from cells expanded on human IgG (control) or on Delta-1^{ext}-IgG, with primers of the indicated specificity (Fzd4 or Dkk2). The bars represent the average expression level relative to TIMM17B±SD (n=3).

### DETAILED DESCRIPTION

Transplantation of myogenic stem cells possesses great potential for long-term repair of dystrophic muscle. However, a single donor muscle biopsy is unlikely to provide enough cells to effectively transplant the muscle mass of a patient affected by muscular dystrophy. Expansion of cells ex vivo using traditional culture techniques significantly reduces engraftment potential. Without wishing to be bound by theory, according to the embodiments described herein it is now believed, based on the present disclosure, that activation of Notch signaling during ex vivo expansion surprisingly maintains donor cell engraftment potential.

As described herein, freshly isolated canine muscle-derived cells were expanded on tissue culture plates coated with Delta-1^{ext}-IgG to activate Notch signaling or with human IgG as a control. A model of canine-to-murine xenotransplantation was used to quantitatively compare canine muscle cell engraftment, and determine if engrafted donor cells could function as satellite cells in vivo. Delta-1^{ext}-IgG inhibited differentiation of canine muscle-derived cells, and increased the level of genes normally expressed in myogenic precursors. Moreover, cells expanded on Delta-1^{ext}-IgG resulted in a significant increase in the number of donor-derived fibers, as compared to cells expanded on human IgG, reaching engraftment levels similar to freshly isolated cells. Importantly, cells expanded on Delta-1^{ext}-IgG engrafted to the recipient satellite cell niche, and contributed to further regeneration.

A similar strategy of expanding human muscle-derived cells on Notch ligand may, according to certain embodiments contemplated herein, thus beneficially facilitate engraftment and muscle regeneration for patients affected with muscular dystrophy. For example, a number of stem cell transplantation and gene therapy approaches are currently under consideration for the treatment of DMD (e.g., Tedesco et al., 2010 J. Clin. Invest. 120:11; Goyenvalle et al., 2011 Hum. Molec. Genet. 20:R69; Tedesco et al., 2011 Sci. Translat. Med. 3:96ra78; Meng et al., 2011 PLoS One 6:e17454; Sacco et al., 2010 Cell 143:1059). These and related approaches may be modified according to the present disclosure, which provides compositions and methods for expanding populations of myogenic precursor cells (MPC) that are present in conventionally obtained skeletal muscle cell preparations, and that can be identified as described herein and according to art-accepted criteria. Engraftment potential is preserved in the MPCs obtained and expanded as described herein, which MPCs may then be administered to a transplantation site according to any of a number of established transplant methodologies, including but not limited to those described, for example, in Tedesco et al., 2010 J. Clin. Invest. 120:11 (and references cited therein); Quattrocelli et al., 2010 Cell Death Diff. 17:1222: Yang et al., 2009 J. Vis. Exp. 31:1388; Perez et al., 2009 Musc. Nerve 40:562; Darabi et al., 2009 Exp. Neurol. 220:212; Markert et al., 2009 PM. R. 1(6):547.

Expansion of myogenic stem cells refers to a statistically significant increase in the myogenic stem cell population, i.e., in the number of stem cells in an in vitro culture, which increase may be achieved through cell division. Expansion may be measured by a doubling in the population of stem cells in the culture, and the rate of population doubling may be used as a measure of the rate of myogenic stem cell expansion. As also noted above, expansion of hematopoietic progenitor cells on Notch ligand maintained their engraftment potential [8-12], and immobilized DII-1 fused to the Fc portion of human IgG (Delta-1^{ext}-IgG) inhibited in vitro differentiation of cultured C2C12 myoblasts [15].

According to certain embodiments described herein, canine muscle-derived cells expanded on immobilized Delta-1^{ext}-IgG were compared to cells expanded on immobilized human IgG control. As described below, activation of Notch signaling during expansion of canine muscle-derived cells inhibited myogenic differentiation. Furthermore, canine-to-mouse xenotransplantation demonstrated that activation of Notch signaling during donor cell expansion maintained engraftment potential. Hence, as described herein it is surprisingly disclosed for the first time that activation-effecting contact with a Notch ligand can maintain myogenic stem cell potential to support muscle cell engraftment.

According to certain further embodiments the present disclosure contemplates optionally contacting a Wnt ligand, or a Wnt ligand receptor agonist, with one or a plurality of MPCs in which Notch signaling is activated as described herein. In such embodiments MPC populations, which have been expanded by Notch activation while preserving engraftment potential as disclosed herein, may be further expanded by activating the canonical and/or non-canonical Wnt signaling pathways. Signal transduction components of the canonical and non-canonical Wnt signaling pathways are well known and may be employed in these and related embodiments based on the present disclosure with no more than routine modification of established methodologies for making and using Wnt ligands and determining canonical and/or non-canonical Wnt signaling pathway activation.

Non-limiting examples of Wnt ligands may include one or more of, e.g., human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16; or a DKK family member such as Dkk-1, Dkk-2 or Dkk-4; or a secreted Frizzled-related protein (sFRP) such as sFRP-1, sFRP-2, sFRP-3, sFRP4 or sFRP-5; Wnt Inhibitory Factor 1 (WIF-1); Norrin; R-spondin; DkkL1; or another recognized Wnt ligand. See, e.g., Nusse et al., 2012 EMBO J. 31:2670; Komiya et al., 2008 Organogen. 4:68; Klaus et al., 2008 Nature Rev. Canc. 8:387; Rao et al., 2010 Circ. Res. 106:1798. Receptors for the Wnt ligands, and Wnt ligand receptor agonists that are capable of activating the canonical or non-canonical Wnt signaling pathway, are also well known and may include, by way of non-limiting example, e.g., FZD1, FZD2, FZD3, FZD4, FZD5, FZD6, FZD7, FZD8, FZD9, LRP5, LRP6, ROR1, ROR2, RYK, MuSK, and/or a glypican such as glypican3. See, e.g., Schulte 2010 Pharmacol. Rev. 62:632; Rao and Kühl, 2010 Circ. Res. 106:1798; Filmus et al., 2008 Genome Biol. 9:224; Chien and Moon, 2007 Front. Biosci. 12:448.

Exemplary Wnt ligands and Wnt ligand receptor agonists are set forth in Table A.

TABLE A
Exemplary Wnt Ligands and Wnt Ligand Receptor Agonists (Genbank Accession Numbers)

| Ligand/ | | | | |
|---|---|---|---|---|
| Agonist | HUMAN | HUMAN | CANINE | CANINE |
| Name | Nucleotide | Protein | Nucleotide | Protein |
| Dkk2 | NM_014421.2 | NP_055236.1 | XM_535681.3 | |
| | | | (predicted) | |
| Fzd4 | NM_012193.3 | NP_036325.2 | XM_843660.2 | XP_848753.2 |
| | | | (predicted) | |
| Wnt1 | NM_005430.3 | NP_005421.1 | XM_543686.3 | XP_543686.3 |
| | | | (predicted) | |
| Wnt2 | NM_003391.2 | NP_003382.1 | XM_849870.1 | XP_854963.1 |
| | | | (predicted) | |
| Wnt2b | NM_004185.3; | NP_078613.1 | XM_540338.3 | ACA13163.1 |
| | NM_024494.2 | | (predicted) | |
| Wnt3 | NM_030753.4 | NP_110380.1 | XM_845071 .2 | XP_850164.1 |
| | | | (predicted) | |
| Wnt3a | NM_033131.3 | NP_149122.1 | XM_539327.3 | XP_539327.3 |
| | | | (predicted) | |
| Wnt4 | NM_030761.4 | NP_110388.2 | XM_850097.3 | XP_855190.2 |
| | | | (predicted) | |
| Wnt5a | NM_003392.4; | NP_003383.2; | XM_541837.3 | XP_541837.3 |
| | NM_001256105.1 | NP_001243034.1 | (predicted) | |
| Wnt5b | NM_032642.2; | NP_110402.2; | XM_543883.3 | XP_543883.3 |
| | NM_030775.2 | NP_116031.1 | (predicted) | |
| Wnt6 | NM_006522.3 | NP_006513.1 | XM_545647.2 | XP_545647.2 |
| | | | (predicted) | |
| Wnt7a | NM_004625.3 | NP_004616.2 | XM_844117.2 | XP_849210.2 |
| | | | (predicted) | |
| Wnt7b | NM_058238.2 | NP_478679.1 | XM_538327.3 | XP_538327.2 |
| | | | (predicted) | |
| Wnt8a | NM_058244.2 | NP_490645.1 | | |
| Wnt8b | NM_003393.3 | NP_003384.2 | XM_543970.2 | XP_543970.2 |
| | | | (predicted) | |
| Wnt9a | NM_003395.2 | NP_003386.1 | XM_539328.2 | XP_539328.2 |
| | | | (predicted) | |
| Wnt9b | NM_003396.1 | NP_003387.1 | XM_548042.3 | XP_548042.3 |
| | | | (predicted) | |
| Wnt10a | NM_025216.2 | NP_079492.2 | XM_545648.3 | XP_545648.2 |
| | | | (predicted) | |
| Wnt10b | NM_003394.3 | NP_003385.2 | XM_543687.2 | XP_543687.2 |
| | | | (predicted) | |
| Wnt11 | NM_004626.2 | NP_004617.2 | XM_542301.3 | XP_542301.2 |
| | | | (predicted) | |
| Wnt16 | NM_057168.1 | NP_476509.1; | XM_850067.2 | XP_855160.2 |
| | | NP_057171.2 | (predicted) | |
| Dkk1 | NM_012242.2 | AAQ89364.1 | XM_846885.2 | XP_851978.2 |
| | | | (predicted) | |
| Dkk2 | NM_014421.2 | AAQ88780.1 | XM_535681.3 | XP_535681.3 |
| | | | (predicted) | |
| Dkk4 | NM_014420.2 | AAI07048.1 | XM_843820.1 | XP_848913.1 |
| | | | (predicted) | |
| sFRP1 | NM_003012.4 | NP_003003.3 | XM_003639564.1 | BAK86425.1 |
| | | | (predicted, partial) | |
| sFRP2 | NM_003013.2 | NP_003004.1 | NM_001002987.1 | NP_001002987.1 |
| sFRP3 | NM_001463.3 | NP_001454.2 | XM_535989.3 | XP_535989.3 |
| (FRZB) | | | (predicted) | |
| sFRP4 | NM_003014.3 | NP_003005.2 | XM_540377.3 | XP_540377.2 |
| | | | (predicted) | |
| sFRP5 | NM_003015.3 | NP_003006.2 | XM_543955.3 | XP_543955.3 |
| | | | (predicted) | |
| WIF-1 | NM_007191.4 | NP_009122.2 | XM_538269.3 | XP_538269.2 |
| | | | (predicted) | |
| Norrin | NM_000266.3 | NP_000257.1 | XM_850168.2 | XP_855261.1 |
| | | | (predicted) | |
| R-spondin | NM_001038633.3; | NP_001033722; | NM_001130838.1 | NP_001124310.1 |
| 1 | NM_001242910.1 | NP_001229837.1 | | |
| | NM_001242908.1; | NP_001229838.1; | | |
| | NM_001242909.1 | NP_001229839.1 | | |
| DKKL1 | NM_001197301.1 | | XM_003638821.1 | XP_003638869.1; |
| | | | (predicted) | XP_864312.1 |
| R-spondin | NM_178565.4 | NP_848660.3 | XM_539125.2 | XP_539125.2 |
| 2 | | | (predicted) | |
| R-spondin | NM_032784.3 | NP_116173.2 | XM_533492.3 | XP_533492.2 |
| 3 | | | (predicted) | |
| R-spondin | NM_001029871.3; | NP_001025042.2; | XM_542937.3 | XP_542937.3 |
| 4 | NM_001040007.2 | NP_001035096.1 | (predicted) | |

Certain presently contemplated embodiments may employ proteins (or encoding polynucleotides therefor) that exhibit structural homology to the herein-disclosed Notch ligands and/or Wnt ligands or Wnt ligand receptor agonists (or encoding polynucleotides therefor). According to non-limiting theory such proteins (or encoding polynucleotides) may be identified by having sequence similarities to the presently disclosed Notch ligands and/or Wnt ligands or Wnt ligand receptor agonists, such as in the amino acid content of and/or spatial distribution of, e.g., charged, neutral and/or hydrophobic amino acids, including exemplary proteins identified by biological sequence database searching (e.g., GenBank, SwissProt, etc.) using sequence database searching software tools as known to the art (e.g., Basic Local Alignment Search Tool (“BLAST”), http://www.ncbi.nlm.nih.gov/BLAST, Altschul, J. Mol. Biol. 219:555-565, 1991, Henikoff et al., Proc. Natl. Acad. Sci. USA 89:10915-10919, 1992; PSI-BLAST, ALIGN, MEGALIGN; WISETOOLS. CLUSTAL W, Thompson et al., 1994 Nucl. Ac. Res. 22:4673; CAP, www.no.embnet. org/clustalw.html; FASTA/FASTP, Pearson, 1990 Proc. Nat. Acad. Sci. USA 85:2444, available from D. Hudson, Univ. of Virginia, Charlottesville, Va.).

Non-limiting examples of such proteins are described herein, any one or more of which may be obtained from the sources as disclosed in the database records and/or synthesized in full or in pertinent part and/or recombinantly expressed in full or in pertinent part (e.g., by selecting a polynucleotide coding region for a peptide fragment having sequence homology to a portion of the desired polypeptide sequence) according to art-established methodologies. (See, e.g., Ausubel et al. (2005 Current Protocols in Molecular Biology, John Wiley & Sons, Inc., Boston, Mass.); Sambrook et al. (2001 Molecular Cloning, Third Ed., Cold Spring Harbor Laboratory, Plainview, N.Y.); Maniatis et al. (1982 Molecular Cloning, Cold Spring Harbor Laboratory, Plainview, N.Y.); Glover (Ed.) (1985 DNA Cloning Vol. I and II, IRL Press, Oxford, UK); Hames and Higgins (Eds.), (1985 Nucleic Acid Hybridization, IRL Press, Oxford, UK). In related embodiments, a wholly synthetic Notch ligand, Wnt ligand or Wnt ligand receptor agonist polypeptide may be generated by chemical synthesis and/or recombinant methodologies, for instance, having an amino acid sequence that is based on a known polypeptide sequence or that is a variant thereof.

Variants may comprise at least 70% sequence identity, preferably at least 75%, 80%, 81%, 82%, 83%, 84%, 85%, 86%, 87%, 88%, 89%, 90%, 91%, 92%, 93%, 94%, 95%, 96%, 97%, 98%, or 99% or higher sequence identity compared to a reference polynucleotide or polypeptide sequence such as the polynucleotide and/or polypeptide sequences disclosed herein (including sequences that are disclosed by reference to Genbank accession numbers), using the methods described herein and known to the art (e.g., BLAST analysis using standard parameters such as the BLASTN 2.0.5 algorithm software described by Altschul et al., Nucleic Acids Res. 1997, 25(17):3389-402, or other similar programs available in the art).

One skilled in this art will recognize that these values can be appropriately adjusted to determine corresponding ability of an encoding polynucleotide to encode a functional ligand by taking into account codon degeneracy, reading frame positioning and the like, and/or to determine the corresponding ability of a Notch ligand polypeptide, a Wnt ligand polypeptide, or a Wnt ligand receptor agonist polypeptide to mediate signaling through a cognate receptor based on conservation of structural features that contribute to effective ligand-receptor engagement, such as known conservative substitutions with regard to amino acid residue charge, polarity (or non-polarity), hydrophobicity, or hydophilicity, or involvement of conserved amino acid residues in a functionally significant structure of the polypeptide such as disulfide bond formation, secondary, tertiary or quarternary structure, glycosylation or other posttranslational modification sites, or the like. Typically, polynucleotide variants will contain one or more substitutions, additions, deletions and/or insertions, preferably such that the signaling ability of the encoded ligand is not substantially diminished relative to that of a Notch ligand polypeptide, a Wnt ligand polypeptide, or a Wnt ligand receptor agonist polypeptide that is specifically set forth herein.

The practice of certain embodiments of the present invention will employ, unless indicated specifically to the contrary, conventional methods in microbiology, molecular biology, biochemistry, molecular genetics, cell biology, virology and immunology techniques that are within the skill of the art, and reference to several of which is made below for the purpose of illustration. Such techniques are explained fully in the literature. See, e.g., Sambrook, et al., Molecular Cloning: A Laboratory Manual (3^{rd} Edition, 2001); Sambrook, et al., Molecular Cloning: A Laboratory Manual (2^{nd} Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3^{rd} Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Riott, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008).

Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

The term “isolated” means that the material is removed from its original environment (e.g., the natural environment if it is naturally occurring). For example, a naturally occurring tissue, cell, nucleic acid or polypeptide present in its original milieu in a living animal is not isolated, but the same tissue, cell, nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons).

Unless the context requires otherwise, throughout the present specification and claims, the word “comprise” and variations thereof, such as, “comprises” and “comprising” are to be construed in an open, inclusive sense, that is, as “including, but not limited to”. By “consisting of” is meant including, and typically limited to, whatever follows the phrase “consisting of.” By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are required and may or may not be present depending upon whether or not they affect the activity or action of the listed elements.

In this specification and the appended claims, the singular forms “a,” “an” and “the” include plural references unless the content clearly dictates otherwise. As used herein, in particular embodiments, the terms “about” or “approximately” when preceding a numerical value indicates the value plus or minus a range of 5%, 6%, 7%, 8% or 9%. In other embodiments, the terms “about” or “approximately” when preceding a numerical value indicates the value plus or minus a range of 10%, 11%, 12%, 13% or 14%. In yet other embodiments, the terms “about” or “approximately” when preceding a numerical value indicates the value plus or minus a range of 15%, 16%, 17%, 18%, 19% or 20%.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### EXAMPLES

### Example 1

### Ex Vivo Expansion of Myogenic Precursors that are Capable of Muscle Engraftment

Materials and Methods:

Donor Cell Isolation.

The Institutional Animal Care and Use Committee at the Fred Hutchinson Cancer Research Center, which is fully accredited by the Association for Assessment and Accreditation of Laboratory Animal Care, approved this study. Elevated enclosed runs were used for housing, and dogs were maintained in social groups wherever possible. All dogs were enrolled in a veterinary preventative medicine program that included a standard immunization series against canine distemper, parvovirus, adenovirus type 2, parainfluenza virus, coronavirus, and rabies.

Each donor canine underwent a maximum of 4 skeletal muscle biopsies. For each canine-to-murine transplantation experiment, a 1 cm×1 cm×0.5 cm skeletal muscle biopsy was harvested from the biceps femoris muscle of the donor canine. The muscle biopsy was trimmed and cut into smaller pieces along the length of the fibers, and digested with 200 U/ml collagenase type 4 (Worthington Biochemical, Lakewood, N.J.) in Dulbecco's Modified Eagle Medium (DMEM; Invitrogen, Carlsbad, Calif.) supplemented with 5 mM CaCl₂, 1 U/ml dispase (Invitrogen), and 0.5% BSA for 30 minutes at 37° C. The intact fibers and muscle pieces were rinsed in Hank's Balanced Salt Solution (HBSS; Invitrogen) and transferred to a new dish. The muscle fibers were chopped and digested fully with 400 U/ml collagenase type I (Sigma-Aldrich, St. Louis, Mo.) in Dulbecco's Modified Eagle Medium (DMEM; Invitrogen) supplemented with 5 mM CaCl₂ for 45 minutes at 37° C. The digested muscle was triturated and filtered through a series of nylon mesh filters. The resulting mononuclear cells released from the muscle were washed twice in PBS, and resuspended in PBS. Mouse muscle-derived cells were isolated using the same method.

Canine Muscle Fiber Isolation.

The muscle biopsies measured approximately 1 cm³, and were from the belly of the canine biceps femoris muscle. We did not remove an entire muscle group tendon-to-tendon, as the biopsy was a survival surgery procedure. Canine muscle biopsies were cut into smaller pieces along the length of the fiber, transferred to Ham's F12 media containing 400 U/ml of collagenase type 1 (Worthington Biochemical), and incubated at 37° C. for 2 hours with regular agitation. The digest was transferred to a 10-cm plate with F12 media supplemented with FBS. The majority of isolated canine muscle fibers appeared hyper-contracted. Fibers of longer length and smoother appearance were visible, yet constituted less than 1% of fibers (data not shown). Using a dissecting microscope, fibers displaying a smooth appearance with no signs of hypercontraction were transferred to PBS using flame-polished pasteur pipettes, and prepared for injection.

Primary Cell Culture.

Each 10-cm tissue culture dish was coated with 50 μg of human IgG (Sigma-Aldrich) or Delta1-1^{ext}^{_{—}}^{Ig} and incubated overnight at 4° C. The following day, the human IgG and Delta-1^{ext}-IgG was removed, and the dishes washed with 1×PBS. The dishes were blocked with 2% bovine serum albumin in 1×PBS for 1 hour at 37° C. After washing the dishes 3× with 1×PBS, canine cells were plated at a density of 7.5×10⁴-1×10⁵ cells per dish in DMEM containing 20% fetal bovine serum and 2.5 ng/ml FGF-2 (Invitrogen). Cells were maintained in culture for 8 days, unless otherwise indicated.

Cells were removed from the dishes by incubating with 5 mM EDTA in Hank's balanced salt solution (HBSS) at 37° C. for 5 minutes. Cells were transferred to a 15-ml conical tube and centrifuged at 1000 rpm for 5 minutes. The cells were washed 3 times, before resuspending in PBS for injection.

Immunocytochemistry.

Primary antibodies specific for Pax7 and myogenin (F5D) were obtained from the Developmental Studies Hybridoma Bank developed under the auspices of the NICHD and maintained by The University of Iowa, Department of Biological Sciences, Iowa City, Iowa. Cultured cells were fixed in 4% paraformaldehyde, and permeabilized with 0.3% Triton X-100 in 1×PBS. Cells were blocked in 10% goat serum, and incubated with primary antibody diluted in primary antibody dilution buffer (1% BSA, 0.1% cold fish skin gelatin, 0.05% sodium azide, 1×PBS) for 1 hour at room temperature. The cells were washed in 1×PBS, incubated with secondary antibody for 1 hour at room temperature, washed with 1×PBS, and mounted with ProLong Gold Anti-fade with DAPI (Invitrogen). Photomicrographs were taken using a Nikon E800 and a CoolSnap camera.

RNA Isolation and RT-qPCR.

RNA was isolated from cells using the RNeasy Kit (Qiagen, Valencia, Calif.) and 1 μg reverse transcribed using SuperScript III (Invitrogen) and random primers. qPCR was performed using an iQ5 machine (BioRad, Hercules, Calif.), using Platinum SYBR Green qPCR SuperMix (Invitrogen), 1/100th of the cDNA reaction mix and the following primers:

[SEQ ID NO: 54]

| | |
|---|---|
| Hey1-F1 | TCGGCTCTAGGTTCCATGTC; |

[SEQ ID NO: 55]

| | |
|---|---|
| Hey1-R1 | AGCAGATCCCTGCTTCTCAA; |

[SEQ ID NO: 56]

| | |
|---|---|
| HeyL-F1 | GATCACTTGAAAATGCTCCAC; |

[SEQ ID NO: 57]

| | |
|---|---|
| HeyL-R1 | TACCTGATGACCTCGGTGAG; |

[SEQ ID NO: 58]

| | |
|---|---|
| Dtx4-F1 | AGCCGCAAAACTACCAAGAA; |

[SEQ ID NO: 59]

| | |
|---|---|
| Dtx-R1 | CGTGAGACGCTCCATACAGA; |

[SEQ ID NO: 60]

| | |
|---|---|
| Pax7-F1 | AAGATTCTCTGCCGCTACCA; |

[SEQ ID NO: 61]

| | |
|---|---|
| Pax7-R1 | TCACAGTGTCCGTCCTTCAG; |

[SEQ ID NO: 62]

| | |
|---|---|
| Myf5-F1 | GGCCTGCCTGAATGTAACAG; |

[SEQ ID NO: 63]

| | |
|---|---|
| Myf5-R1 | GTTGCTCGGAGTTGGTGATT; |

[SEQ ID NO: 64]

| | |
|---|---|
| musculin-F1 | GGCTGGCATCCAGTTACATC; |

[SEQ ID NO: 65]

| | |
|---|---|
| musculin-R1 | GCGGAAACTTCTTTGGTGTC; |

[SEQ ID NO: 66]

| | |
|---|---|
| MyoD-F1 | CGATTCGCTACATCGAAGGT; |

[SEQ ID NO: 67]

| | |
|---|---|
| MyoD-R1 | AGGTGCCATCGTAGCAGTTC; |

[SEQ ID NO: 68]

| | |
|---|---|
| CXCR4-F1 | GAGCTCCATATATACCCTTCAGATA; |

[SEQ ID NO: 69]

| | |
|---|---|
| CXCR4-R1 | GGTAACCCATGACCAGGATG; |

[SEQ ID NO: 70]

| | |
|---|---|
| CD34-F1 | TGACCCAAGTCCTGTGTGAG; |

[SEQ ID NO: 71]

| | |
|---|---|
| CD34-R1 | GTCTTGCGGGAATAGCTCTG; |

[SEQ ID NO: 72]

| | |
|---|---|
| cadherin11-F1 | GAACCAGTTCTTCGTGATAGAGGA; |

[SEQ ID NO: 73]

| | |
|---|---|
| cadherin11-R1 | TGTCTTGGTGGCATGAATGT; |

[SEQ ID NO: 74]

| | |
|---|---|
| TIMM17B-F1 | ATCAAGGGCTTCCGCAATG; |

[SEQ ID NO: 75]

| | |
|---|---|
| TIMM17B-R1 | CACAGTCGATGGTGGAGAACAG. |

Threshold cycle values were used to generate relative gene specific expression values normalized to TIMM17B expression. To confirm accuracy, the data were also normalized to expression of TBP.

Fluorescence Activated Cell Sorting (FACS).

Anti-CXCR4 was obtained from R & D Systems (clone 44716; Minneapolis, Minn.) and used at 10 μg/ml for FACS sorting of 1×10⁶ cells. Anti-syndecan 4 and Alexa Fluor 488 labeled anti-chicken antibody were kind gifts of D. D. Cornelison (University of Missouri). Alexa Fluor 488-labeled anti-mouse IgG2b was obtained from Invitrogen (Carlsbad, Calif.) and used at 1:200. Expanded canine skeletal muscle cells dissociated from the plate were resuspended in FACS buffer (Hanks Balanced Salt Solution [HBSS], 5% FBS) and incubated on ice with anti-CXCR4, anti-syndecan 4 or isotype control, followed by Alexa Fluor 488-labeled secondary antibodies. The cells were washed, resuspended in FACS buffer, and sorted using a FACSCalibur (BDBiosciences, Franklin Lakes, N.J.).

Cell Injection into Mice and Tissue Processing.

The right hindlimb of each 7-12 week old NOD/SCID mouse was exposed to 12 Gy of ionizing irradiation (Mark 1 cesium source, Sheppard and Associates), and the tibialis anterior (TA) muscle of the same hindlimb was injected with 50 μl of 1.2% barium chloride immediately after irradiation. The following day, the same TA muscle was injected with 50 μl of freshly isolated canine muscle-derived cells or mouse muscle-derived cells, or cells expanded on human IgG or Delta-1^{ext}-IgG, along the length of the muscle, so as to distribute cells from the distal to the proximal end of the muscle. The injected muscle was harvested 28 days after injection, unless otherwise indicated.

The harvested mouse muscle was covered in OCT within a plastic cryomold and placed on top of an aluminum block immersed in liquid nitrogen. Frozen tissue was stored at −80° C. Cryosections were cut (10 μm) from the distal to the proximal end of the frozen muscle using a Leica CM1850 cryostat, and adhered to Superfrost slides (Fisher Scientific). Each glass slide consisted of 4 serial sections, and the corresponding section on the subsequent slide represented a separation of approximately 200 μm from the previous slide.

Each TA muscle normally generated 24 slides, each consisting of 4 serial sections. Initially, slides 6, 12, and 18 were stained for dystrophin and lamin A/C to determine the region of highest engraftment. Three more even numbered slides were chosen from the region of highest engraftment and stained for canine dystrophin and lamin A/C. Three odd numbered slides in the same region were used for Pax7 and lamin A/C co-staining. In almost all cases, the region of highest engraftment was between slides 6 and 18, representing the belly of the muscle, which does not vary considerably in cross-sectional area.

Immunostaining.

Anti-dystrophin (MANDYS107) was obtained from the Developmental Studies Hybridoma Bank developed under the auspices of the NICHD and maintained by The University of Iowa, Department of Biological Sciences, Iowa City, Iowa. Anti-lamin A/C (clone 636) and anti-developmental myosin heavy chain were obtained from Vector Laboratories (Burlingame, Calif.). Alexa fluor 488 conjugated goat anti-mouse IgG and Alexa fluor 568-conjugated goat anti-mouse IgG2b secondary antibodies, both from Invitrogen, were used at 1:200. For dystrophin and lamin A/C staining, the sections were fixed in acetone at −20° C. for 10 minutes, allowed to dry, and rehydrated in PBS. Sections were incubated in blocking buffer (2% goat serum, 1% BSA, 0.1% cold fish skin gelatin, 0.05% sodium azide, 1×PBS) for 1 hour at room temperature, followed by primary antibody diluted in primary antibody dilution buffer (1% BSA, 0.1% cold fish skin gelatin, 0.05% sodium azide, 1×PBS) for 1 hour at room temperature, or overnight at 4° C. The sections were washed in 1×PBS, incubated with secondary antibody for 1 hour at room temperature, washed with 1×PBS, and mounted with ProLong Gold Anti-fade with DAPI (Invitrogen).

Primary antibody specific for Pax7 antibody was used at 1:10, and was obtained from the Developmental Studies Hybridoma Bank. Alexa fluor-conjugated goat anti-mouse IgG1 (Pax7), Alexa fluor 568 conjugated goat anti-mouse IgG2b (lamin A/C) was used at 1:200, and was obtained from Invitrogen. For Pax7 and lamin A/C co-staining, cryosections were fixed in 4% paraformaldehyde for 20 minutes at room temperature, washed with 1×PBS, followed by permeabilization with methanol at −20° C. for 6 minutes. The sections were washed in 1×PBS, and antigen retrieval was performed by incubating the slides twice in 10 mM citric acid (pH 6.0) at 90° C. for 5 minutes. Sections were washed with 1×PBS, blocked in blocking buffer (2% goat serum, 1% BSA, 0.1% cold fish skin gelatin, 0.05% sodium azide, 1×PBS) for 1 hour at room temperature, and incubated in primary antibody diluted in primary antibody dilution buffer (1% BSA, 0.1% cold fish skin gelatin, 0.05% sodium azide, 1×PBS) for 1 hour at room temperature, or overnight at 4° C. The sections were washed in 1×PBS, incubated with secondary antibody for 1 hour at room temperature, washed with 1×PBS, and mounted with ProLong Gold Anti-fade with DAPI (Invitrogen).

Photomicrographs were taken using a Zeiss Axiolmager.Z1 as part of a TissueFaxs system (TissueGnostics, Los Angeles, Calif.). The images for each field of view were stitched together to form an entire cross-sectional view. The number of fibers expressing canine dystrophin, the number of nuclei expressing canine lamin A/C, and the number of nuclei expressing canine lamin A/C and Pax7 were counted from these cross-sectional views.

Results:

Expanding Canine Muscle Cells Negatively Impacted Engraftment.

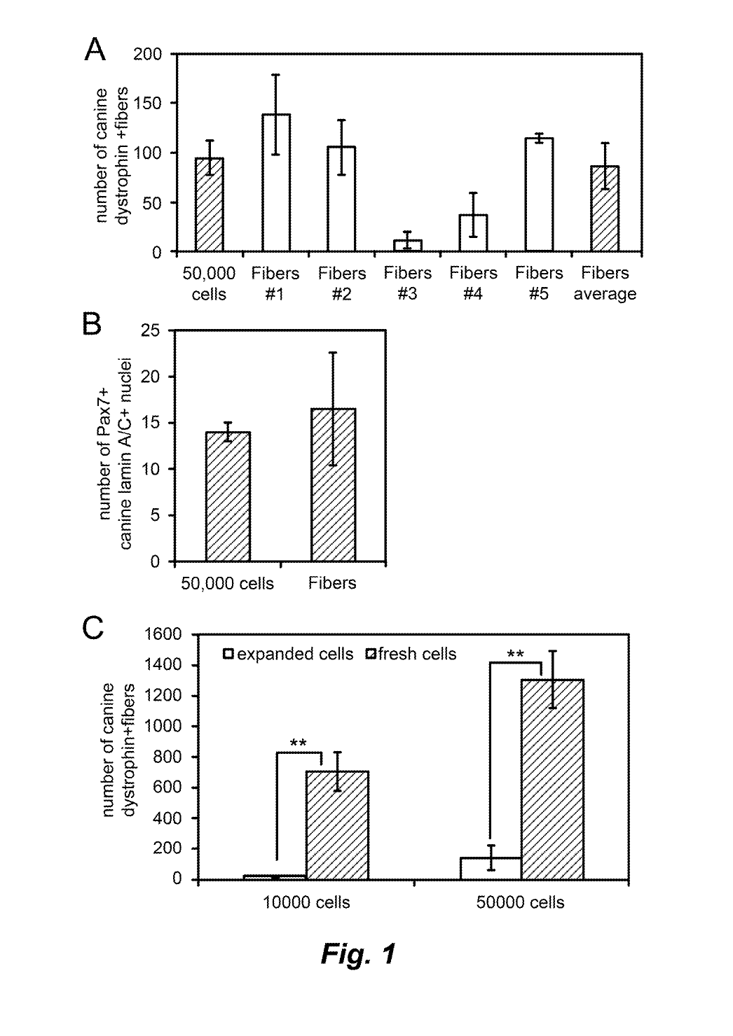

Currently, muscle fiber preparations and freshly isolated muscle-derived cells are considered the most effective material for muscle transplantation. To compare the engraftment efficiency of fresh fibers to freshly isolated muscle-derived cells, we transplanted each population into the tibialis anterior muscle of a NOD/SCID mouse, as previously described [17]. The mouse hindlimb was pre-irradiated with 12 Gy of ionizing radiation to prevent regeneration by host mouse satellite cells and pre-treated with BaCl₂ to induce muscle degeneration (see Methods). On average, injection of 50,000 freshly isolated canine muscle-derived cells appeared to be equivalent to injection of 5 single canine muscle fibers from the same donor muscle biopsy, comparing both the number of fibers expressing canine dystrophin and the number of nuclei expressing Pax7 and canine lamin A/C (FIG. 1A, 1B). Because each isolated muscle fiber might have approximately ten mononuclear cells capable of regeneration, our results are consistent with prior studies showing that transplanting muscle fibers shows the greatest per cell regeneration potential [6,7].

Despite the superior potential, muscle fiber preparations are not likely to yield enough transplantable material to treat all muscles of an individual affected with muscular dystrophy. Therefore, to achieve sufficient numbers of donor cells for large scale transplantation, ex vivo expansion will be required. However, muscle-derived cells expanded in vitro on standard tissue culture dishes displayed significantly reduced engraftment as compared to freshly isolated cells (FIG. 1C).

The donor used for the experiment in FIG. 1C was not the same donor used for the experiment in FIG. 1A. Therefore, the difference in the level of engraftment observed between FIGS. 1A and 1C likely reflects how each donor's muscle-derived cell population has a different capacity for reconstitution [17]. Moreover, the freshly isolated cells transplanted for the experiment in FIG. 1A remained on ice for a longer period of time before transplant to accommodate the muscle fiber preparation, which may have had a negative impact on engraftment.

Yet, these results are consistent with previous studies showing that expanding myoblasts in vitro diminishes transplantation efficiency [4,5]. Based on studies of the in vitro expansion of hematopoietic stem cells, we hypothesized that activating Notch signaling in muscle-derived cells during expansion would maintain engraftment potential of donor cells.

Activation of Notch Signaling Inhibited Canine Myogenic Differentiation.

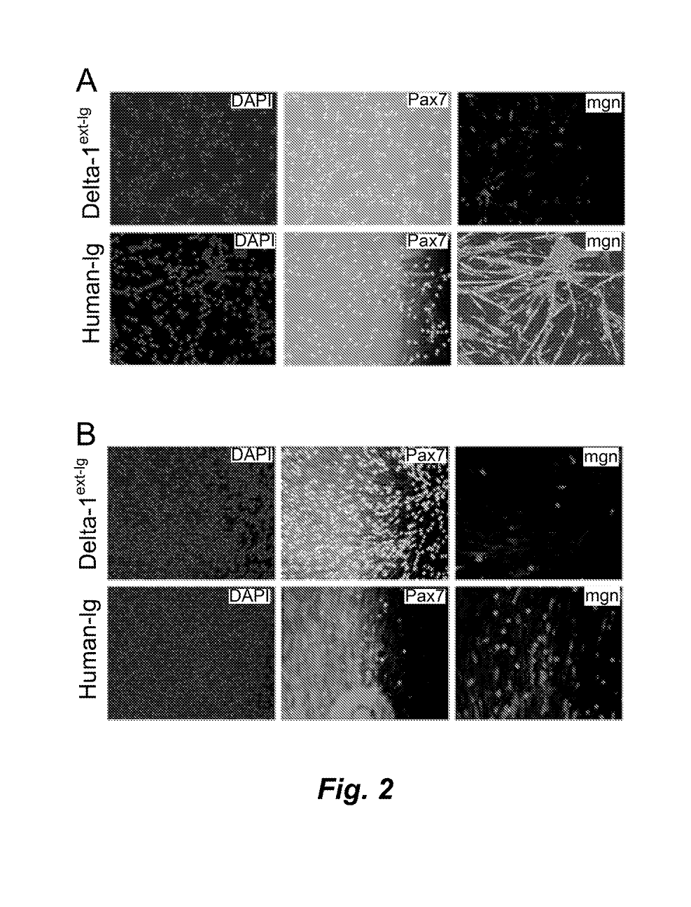

To mimic activation of Notch signaling, tissue culture treated polystyrene plates were coated with Delta1^{ext}-IgG. Control plates were coated with human IgG. Canine satellite cell-derived myoblasts, previously cultured on uncoated tissue culture plates, were cultured on Delta-1^{ext}-IgG or human Ig coated plates for 8 days in DMEM supplemented with 20% FBS and 2.5 ng/ml FGF. As predicted by studies with mouse myoblasts, Delta-1^{ext}-IgG inhibited differentiation of canine myoblasts (FIG. 2A).

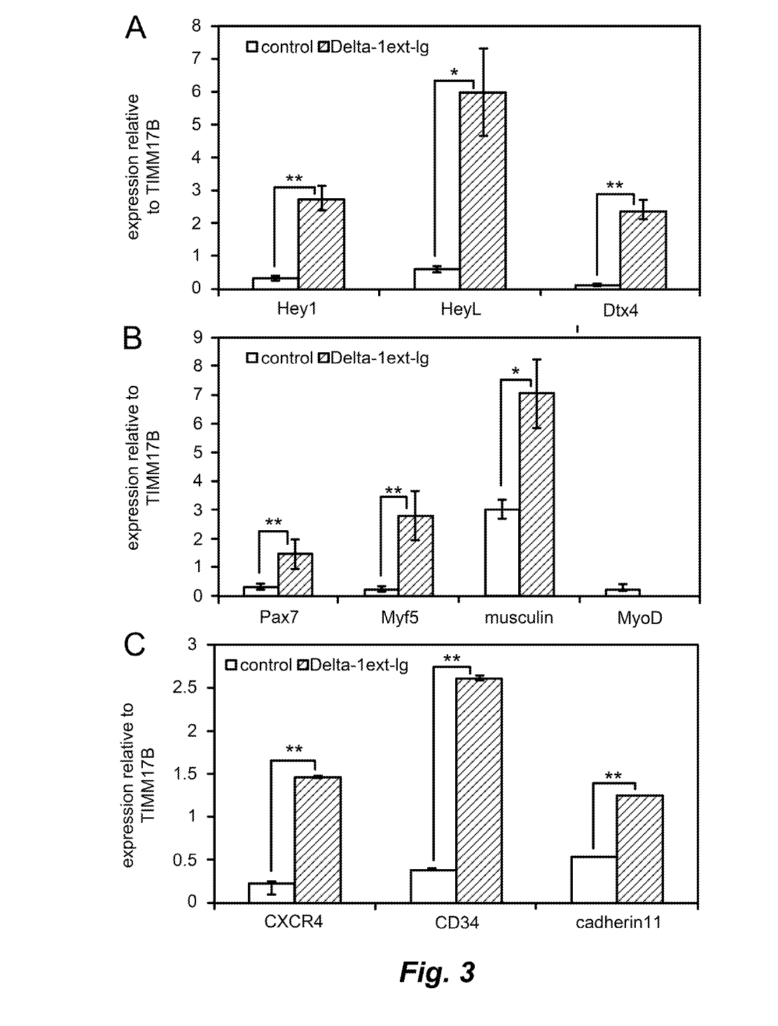

Similarly, exposure of freshly isolated canine muscle-derived cells to Delta-1^{ext}-IgG inhibited differentiation (FIG. 2B), and resulted in a 6.5- to 20-fold expansion of total cell number over 8 days (Table 2). Increased expression of Hey1, HeyL, and Dtx4 confirmed activation of Notch signaling in cells exposed to Delta-1^{ext}-IgG (FIG. 3A).

Expression of musculin, an inhibitor of myogenic differentiation, was significantly increased in cells exposed to Delta-1^{ext}-IgG. This was accompanied by a significant decrease in expression of MyoD, and an increase in expression of Myf5 and Pax7 in cells expanded on Delta-1^{ext}-IgG (FIG. 3B). Expression of myogenin was almost undetectable in cells grown on human IgG, but completely absent from cells grown on Delta-1^{ext}-IgG (data not shown), confirming immunocytochemistry results (see FIG. 2). Therefore, Delta-1^{ext}-IgG inhibited canine myogenic differentiation.

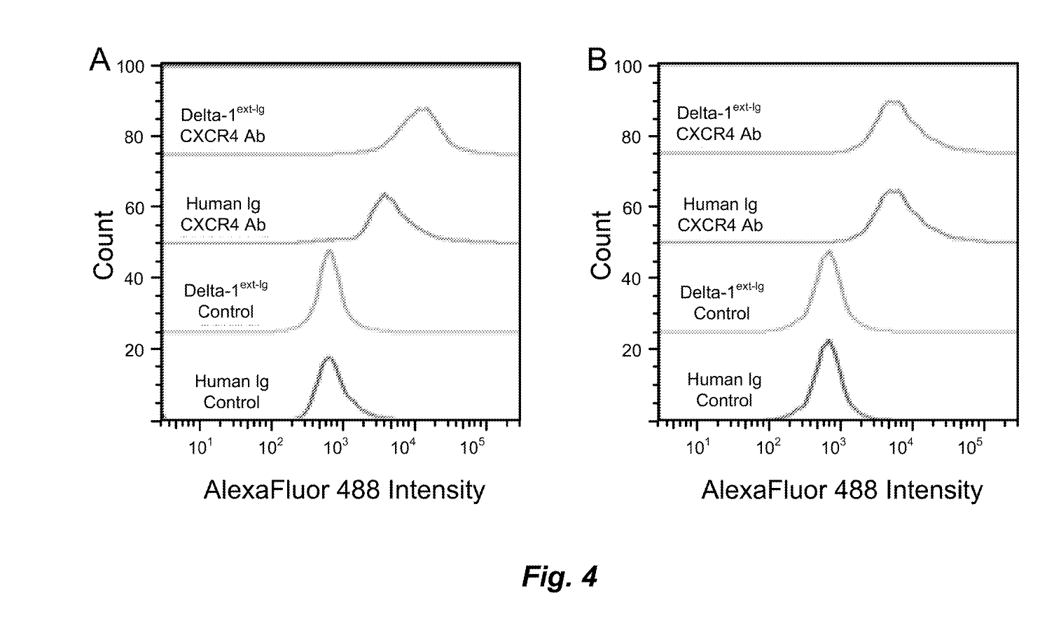

When compared to cells expanded on human IgG, expanding cells on Delta-1^{ext}-IgG did not increase the percentage of cells expressing syndecan 4, a marker of satellite cells and satellite cell-derived myogenic cells in culture (FIG. 4B)(Table 1) [16].

TABLE 1
Effect of Expanding cells on Delta-1^{ext}-IgG on the number of CXCR4⁺ or
syndecan 4⁺ cells.

| | isotype | | isotype | |
|---|---|---|---|---|
| | control | CXCR4 | control | syndecan 4 |
| Human | 0.74% | 78.6% | 1.17% | 89.5% |
| IgG | | | | |
| Delta-1^{ext}- | 0.78% | 81.5% | 3.25% | 81.1% |
| IgG | | | | |

The percent of Alexa Fluor 488-positive cells were determined from the FACS sort shown in FIG. 4.

In contrast, the CXCR4 receptor, which has a critical role in muscle regeneration [17], showed increased RNA and protein levels in cells expanded on Delta-1^{ext}-IgG (FIGS. 3C and 4A), however, the percentage of CXCR4 expressing cells did not increase (Table 1), indicating a higher abundance of CXCR4 per cell. Together, these data show that culture of primary muscle-derived cells on Delta-1^{ext}-IgG promotes the expansion of Pax7 and Myf5 positive cells with enhanced CXCR4 expression.

TABLE 2
Expansion of canine muscle derived cells.

| | | cell number | cell number |
|---|---|---|---|
| | | final | final |
| | cell number | Delta-1^{ext}-IgG | human IgG |
| Experiment | start | (fold-increase) | (fold-increase) |
| 1 | 7.5 × 10⁴ | 1.5 × 10⁶ | 2.2 × 10⁶ |
| | | (20) | (29.3) |
| 2 | 1 × 10⁵ | 6.5 × 10⁵ | 1.1 × 10⁶ |
| | | (6.5) | (11) |
| 3 | 1 × 10⁵ | 8.4 × 10⁵ | 2.6 × 10⁶ |
| | | (8.4) | (26) |

Freshly isolated canine muscle-derived cells were cultured on plates coated with Delta-1^{ext}-IgG or human IgG. After 8 days, the cells were dissociated from the plates, pooled, and the number of cells per plate determined. The final cell numbers represent the average of 2 (Experiment 1), 7 (Experiment 2), or 6 (Experiment 3) 10-cm culture plates.

Activation of Notch Signaling During Expansion Maintained Engraftment of Donor Cells.

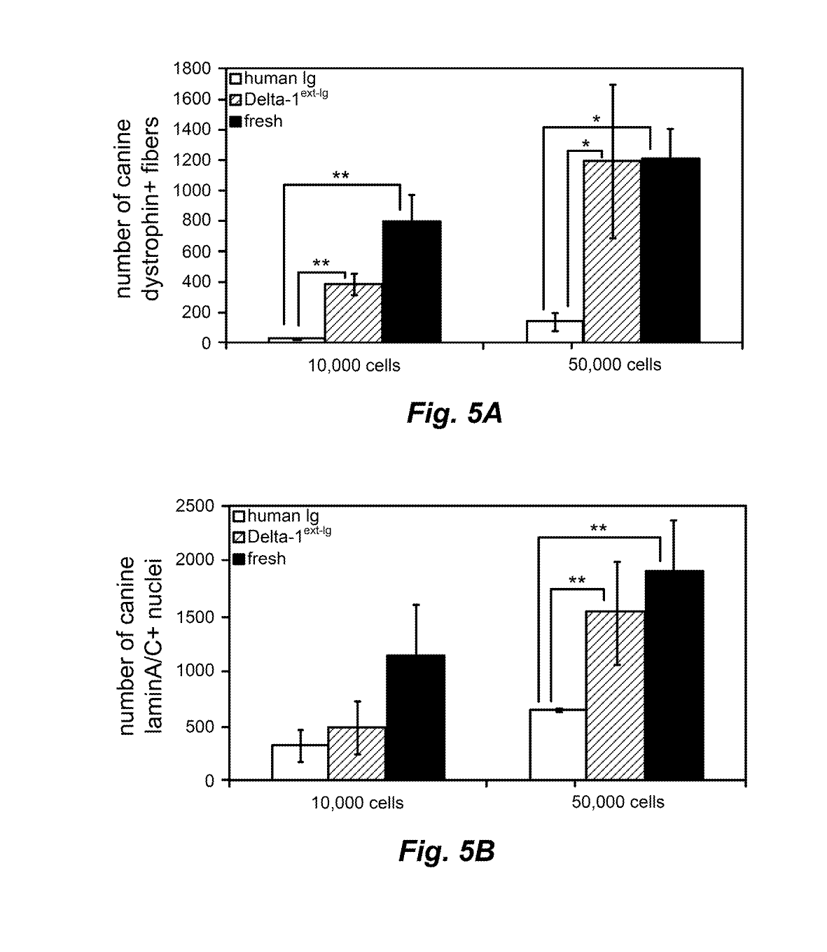

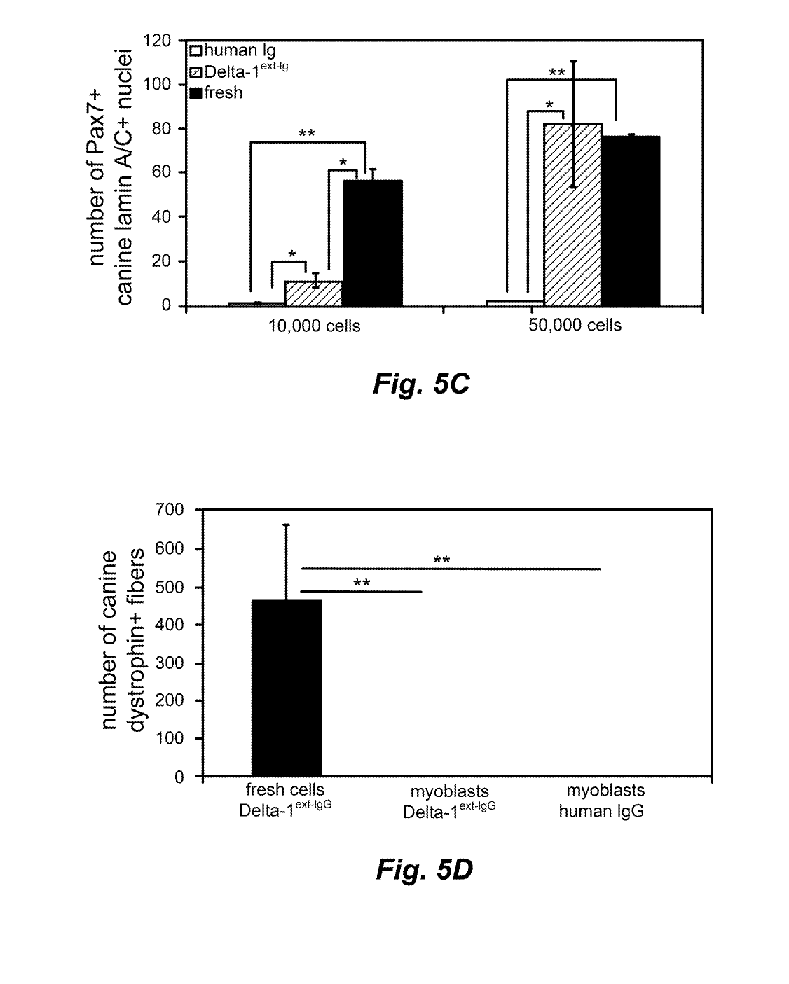

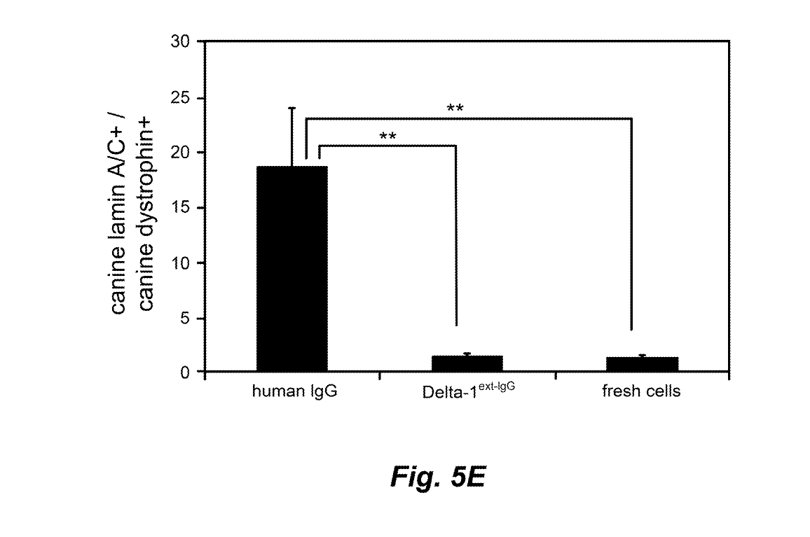

Engraftment of 5×10⁴ cells expanded on Delta-1^{ext}-IgG was similar to engraftment of 5×10⁴ freshly isolated cells, as shown by the similar number of fibers expressing canine dystrophin, nuclei expressing canine lamin A/C, and nuclei expressing canine lamin A/C and Pax7 (FIG. 5A-C). Approximately 80% of cells expanded on Delta-1^{ext}-IgG are myogenic cells, as evidenced by syndecan 4 expression (Table 1), whereas, less than 4% of freshly isolated cells generate myogenic cell clones in culture (data not shown).

In contrast, transplantation of cells expanded on human IgG resulted in significantly fewer fibers expressing canine dystrophin and less than 1 nuclei co-expressing Pax7 and canine lamin A/C per cross-section, similar to cells expanded on uncoated tissue culture plates (see FIG. 1C). Therefore, Notch activation during in vitro muscle cell expansion maintained engraftment potential. However, muscle-derived cells must be exposed to Delta-1^{ext}-IgG immediately after isolation, as activating Notch activity in myoblasts previously cultured on uncoated tissue culture plates did not restore engraftment potential (FIG. 5D).

The enhanced muscle regeneration capacity of muscle cells expanded on the Notch ligand was largely due to enhanced myogenesis rather than simple cell survival, based on the ratio of donor lamin A/C+ cells to donor myofibers (FIG. 5E). For muscle injected with cells expanded on human IgG, the ratio of the number of canine lamin A/C-positive nuclei to the number of canine dystrophin-positive fibers per cross-section was 18.6; however, the ratio is 1.7 for muscle injected with cells expanded on Delta-1^{ext}-IgG, and 1.8 for muscle injected with fresh cells. This indicates that cells expanded on human IgG survived transplantation but did not contribute as effectively to the formation of fibers expressing canine dystrophin during regeneration as compared to cells expanded on Delta-1^{ext}-IgG or fresh cells.

Expanded Cells Contribute to Further Regeneration.

The presence of Pax7+ donor canine cells suggests that some donor cells enter a repopulating or satellite cell compartment. To determine whether the engrafted donor muscle cells are capable of regeneration, mice were subjected to two additional rounds of intramuscular BaCl₂ injection at 4 and 8 weeks after donor cell transplant. As noted above, the initial hindlimb irradiation prior to the donor cell transplantation prevents muscle regeneration from the host mouse satellite cells and the majority of muscle repair will require donor canine satellite cell activity.

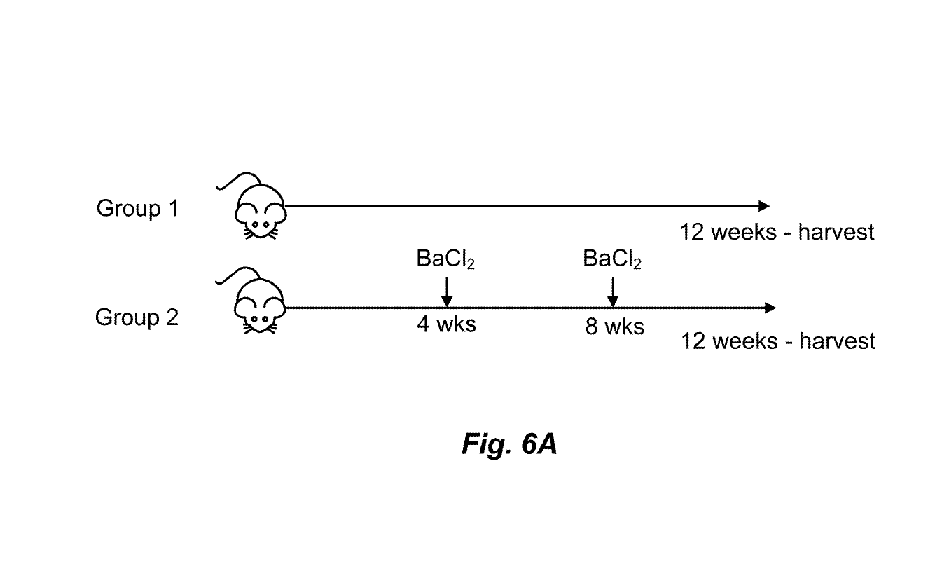

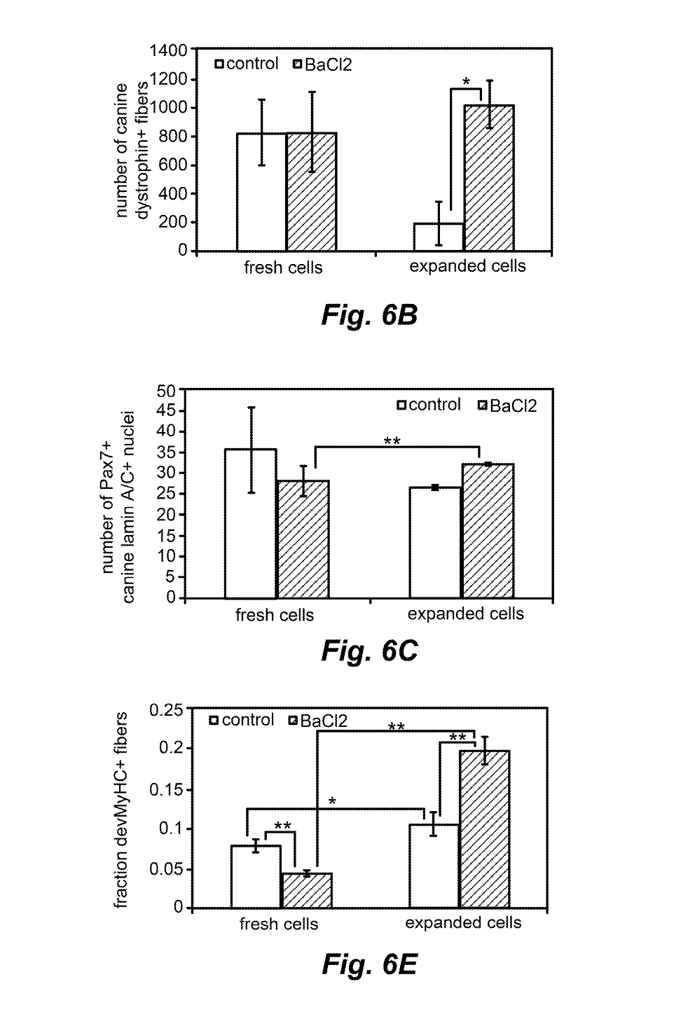

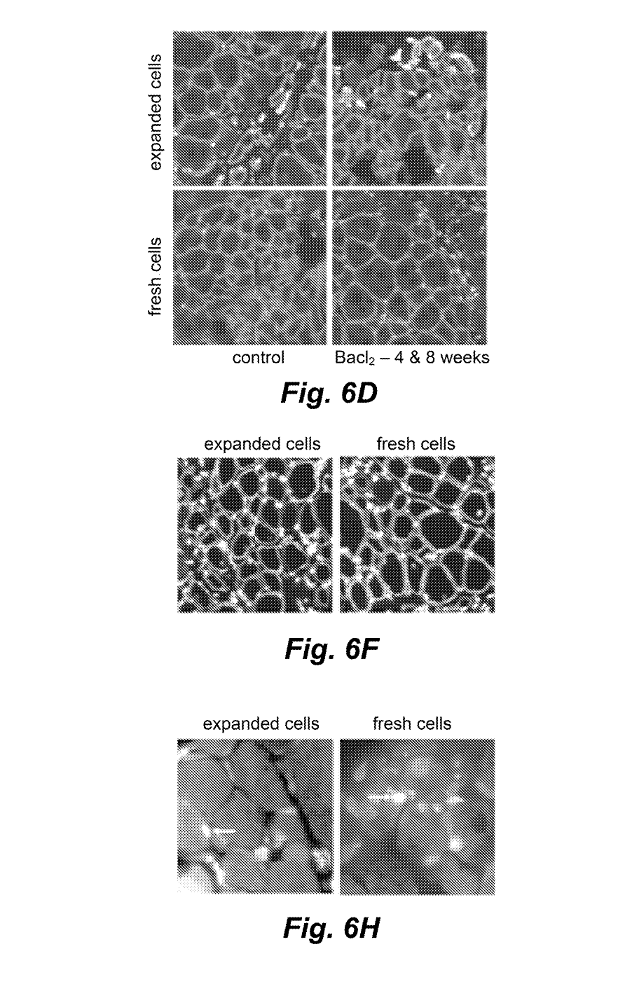

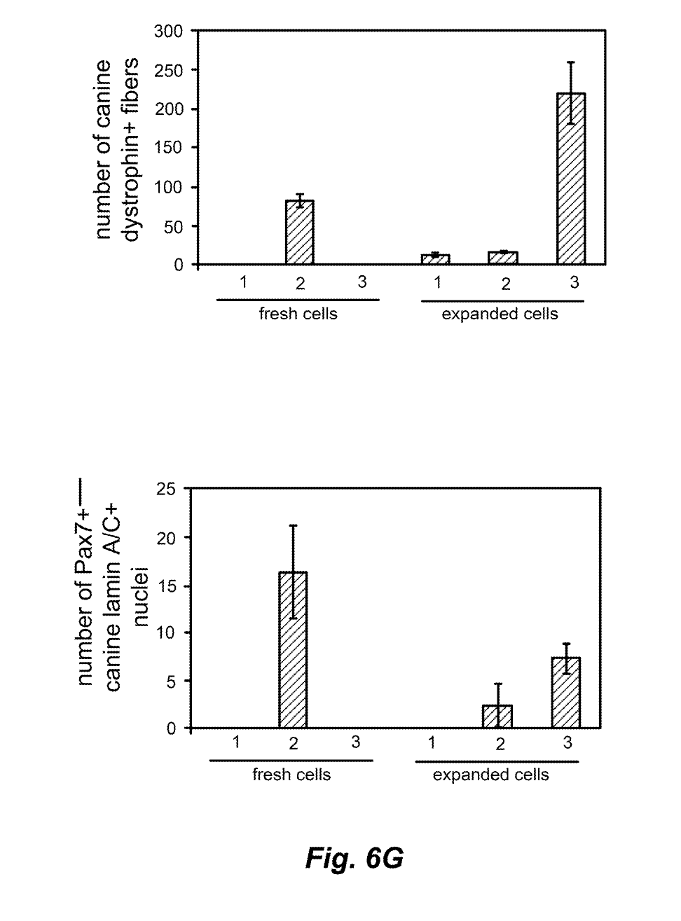

Four weeks following two additional rounds of BaCl₂-induced regeneration, muscle injected with Delta-1^{ext}-IgG expanded cells showed a significant increase in the number of fibers expressing canine dystrophin and a consistent number of nuclei co-expressing Pax7 and canine lamin A/C (FIG. 6A-C). Expression of a developmental form of myosin heavy chain (devMyHC), expressed in immature myofibers, indicated ongoing muscle regeneration (FIGS. 6 D and E).

To further demonstrate the ability of engrafted cells to participate in regeneration, we performed secondary transplants using cells isolated from mouse muscle injected with freshly isolated canine muscle-derived cells, or Delta-1^{ext}-IgG expanded cells. All three secondary recipients of Delta-1^{ext}-IgG expanded cells displayed fibers expressing canine dystrophin, and nuclei co-expressing Pax7 and canine lamin A/C were detected in two recipients (FIG. 6F-I). However, there was no statistically significant difference in the level of engraftment between secondary recipients of fresh cells and Delta-1^{ext}-IgG expanded cells.

Together these data indicate that canine donor cells expressing Pax7 in muscle transplanted with cells expanded on Delta-1^{ext}-IgG can function in a manner similar to satellite cells and participate in muscle regeneration, and maintain a Pax7⁺ population after regeneration.

Discussion

The number of myogenic cells was not significantly different between cells expanded on Delta-1^{ext}-IgG and cells expanded on human IgG; however, Pax7 expression was increased in canine cells expanded on Delta-1^{ext}-IgG. This suggests that upregulating Notch activity during ex vivo expansion increased the number of myogenic progenitor cells that are similar to quiescent or newly activated satellite cells.

Activation of Notch signaling in canine muscle-derived cells resulted in downregulation of MyoD and myogenin expression [13, 14], and an increase in Myf5, Pax7, and CXCR4 expression. Myf5 was not expressed during myogenic differentiation [22, 23], and Myf5 transcripts have been detected in quiescent and newly activated satellite cells [24-27]. Increased expression of Myf5 indicates that induction of Notch signaling with Delta-1^{ext}-IgG during in vitro culture of the canine muscle-derived cells resulted in maintenance and expansion of a myogenic cell with characteristics of an early activated satellite cell.

Blocking CXCR4 receptor activity on donor cells before transplant significantly impaired donor cell engraftment [17]. In contrast, promoting CXCR4 activity by inhibiting CD26/DPP-IV degradation of SDF-1 with diprotin A enhanced donor cell engraftment. Together, these observations suggest that CXCR4 may be a marker of donor cells that effectively participate in donor cell dependent muscle regeneration. Increased expression of CXCR4 in cells expanded on Delta-1^{ext}-IgG may provide part of the reason for the increase in engraftment compared to cells expanded on human IgG, indicating that diprotin A may have a potent effect on engraftment of cells expanded on Delta-1^{ext}-IgG.

In hematopoietic transplant, short-term repopulating cells are more committed progenitors that engraft quickly; however long-term repopulating cells are more primitive cells capable of self-renewal. BaCl₂-induced regeneration in muscle transplanted with canine cells expanded on Delta-1^{ext}-IgG increased the number of fibers expressing canine dystrophin, and maintained the number of donor Pax7⁺ cells. Moreover, engraftment was detected in secondary recipients of Delta-1^{ext}-IgG expanded cells. Donor cells expanded on Delta-1^{ext}-IgG that had engrafted into recipient muscle thus participated in muscle repair similar to satellite cells, and had the capacity to self-renew, similar to long-term repopulating hematopoietic cells. Together, these data suggest according to non-limiting theory that activating Notch signaling during expansion of canine muscle-derived cells maintained a subpopulation of progenitor cells.

Effective expansion of cells ex vivo for transplant may involve mimicking the fiber environment, both biophysically and biochemically, to maintain a large proportion of cells as stem cells. The ability to expand donor muscle-derived cells ex vivo may therefore represent an important step towards making cell transplantation a therapeutic option for muscular dystrophies. Similarly, immobilized Delta-1^{ext}-IgG inhibits differentiation of human CD34⁺CD38-cord blood precursors, and dramatically increases the number of precursors capable of repopulating NOD/SCID mice [8, 9, 11]. A phase 1 clinical trial of transplantation of ex vivo expanded CD34⁺CD38-cord blood precursors is currently underway in patients with high risk leukemias, and appears to successfully promote donor cell engraftment [12]. According to the present disclosure, a strategy of expanding human muscle-derived cells on Notch ligand may facilitate engraftment and muscle regeneration and thus may provide effective avenues for human muscle transplantation.

### REFERENCES

1 Partridge T A, Morgan J E, Coulton G R et al. Conversion of mdx myofibres from dystrophin-negative to -positive by injection of normal myoblasts. Nature 1989; 337:176-179.
2 Karpati G, Pouliot Y, Zubrzycka-Gaarn E et al. Dystrophin is expressed in mdx skeletal muscle fibers after normal myoblast implantation. Am J Pathol 1989; 135:27-32.
3 Huard J, Labrecque C, Dansereau G et al. Dystrophin expression in myotubes formed by the fusion of normal and dystrophic myoblasts. Muscle Nerve 1991; 14:178-182.
4 Parker M H, Kuhr C, Tapscott S J et al. Hematopoietic cell transplantation provides an immune-tolerant platform for myoblast transplantation in dystrophic dogs. Mol Ther 2008; 16:1340-1346.
5 Montarras D, Morgan J, Collins C et al. Direct isolation of satellite cells for skeletal muscle regeneration. Science 2005; 309:2064-2067.
6 Collins C A, Olsen I, Zammit P S et al. Stem cell function, self-renewal, and behavioral heterogeneity of cells from the adult muscle satellite cell niche. Cell 2005; 122:289-301.
7 Hall J K, Banks G B, Chamberlain J S et al. Prevention of muscle aging by myofiber-associated satellite cell transplantation. Science Translational Medicine 2010; 2: 57ra83.
8 Ohishi K, Varnum-Finney B, Bernstein I D. Delta-1 enhances marrow and thymus repopulating ability of human CD34⁺CD38⁻ cord blood cells. J Clin Invest 2002; 110:1165-1174.
9 Varnum-Finney B, Brashem-Stein C, Bernstein I D. Combined effects of Notch signaling and cytokines induce a multiple log increase in precursors with lymphoid and myeloid reconstituting ability. Blood 2003; 101:1784-1789.
10 Delaney C, Varnum-Finney B, Aoyama K et al. Dose-dependent effects of the Notch ligand Delta1 on ex vivo differentiation and in vivo marrow repopulating ability of cord blood cells. Blood 2005; 106:2693-2699.
11 Dallas M H, Varnum-Finney B, Martin P J et al. Enhanced T-cell reconstitution by hematopoietic progenitors expanded ex vivo using the Notch ligand Delta1. Blood 2007; 109:3679-3587.
12 Delaney C, Heimfeld S, Brashem-Stein C et al. Notch-mediated expansion of human cord blood progenitor cells capable of rapid myeloid reconstitution. Nat Med 2010; 16:232-237.
13 Conboy I M, Rando T A. The regulation of Notch signaling controls satellite cell activation and cell fate determination in postnatal myogenesis. Developmental Cell 2002; 3:397-409.
14 Kopan R, Nye J S, Weintraub H. The intracellular domain of mouse Notch: a constitutively activated repressor of myogenesis directed at the basic helix-loop-helix region of MyoD. Development 1994; 120:2385-2396.
15 Varnum-Finney B, Wu L, Yu M et al. Immobilization of Notch ligand, Delta-1, is required for induction of Notch signaling. J Cell Sci 2000; 113:4312-4318.
16 Berg Z, Beffa L R, Cook D P et al. Muscle satellite cells from GRMD dystrophic dogs are not phenotypically distinguishable from wild type satellite cells in ex vivo culture. Neuromuscular Disorders 2011; 21:282-290.
17 Parker M H, Loretz C, Tyler A et al. Inhibition of CD26/DPP-IV enhances donor muscle cell engraftment and stimulates sustained donor cell proliferation. Skeletal Muscle 2012; 2:4.
18 Gilbert P M, Havenstrite K L, Magnusson K E et al. Substrate elasticity regulates skeletal muscle stem cell self-renewal in culture. Science 2010; 329:1078-1081.
19 Engler A J, Griffin M A, Sen S et. al.. Myotubes differentiate optimally on substrates with tissue-like stiffness: pathological implications for soft or stiff microenvironments. J Cell Biol 2004; 166:877-887.
20 Mourikis P, Sambasivan R, Castel D et al. A critical requirement for notch signaling in maintenance of the quiescent skeletal muscle stem cell state. Stem Cells 2012; 30:243-252.
21 Bjornson C R, Cheung T H, Liu L et al. Notch signaling is necessary to maintain quiescence in adult muscle stem cells. Stem Cells 2012; 30:232-242.
22 Lindon C, Montarras D, Pinset C. Cell cycle-regulated expression of the muscle determination factor Myf5 in proliferating myoblasts. J Cell Biol 1998; 140:111-118.
23 Yablonka-Reuveni Z, Rudnicki M A, Rivera A J et al. The transition from proliferation to differentiation is delayed in satellite cells from mice lacking MyoD. Dev Biol 1999; 210:440-455.
24 Beauchamp J R, Heslop L, Yu D S et al. Expression of CD34 and Myf5 defines the majority of quiescent adult skeletal muscle satellite cells. J Cell Biol 2000; 151:1221-1234.
25 Zammit P S, Relaix F, Nagata Y et al. Pax7 and myogenic progression in skeletal muscle satellite cells. J Cell Sci 2006; 119:1824-1832.
26 Day K, Shefer G, Richardson J B et al. Nestin-GFP reporter expression defines the quiescent state of skeletal muscle satellite cells. Dev Biol 2007; 304:246-259.
27 Cornelison D D, Wold B J. Single-cell analysis of regulatory gene expression in quiescent and activated mouse skeletal muscle satellite cells. Dev Biol 1997; 191:270-283.

### Example 2

### Upregulation of Wnt Signaling Pathway Components by Notch Activation

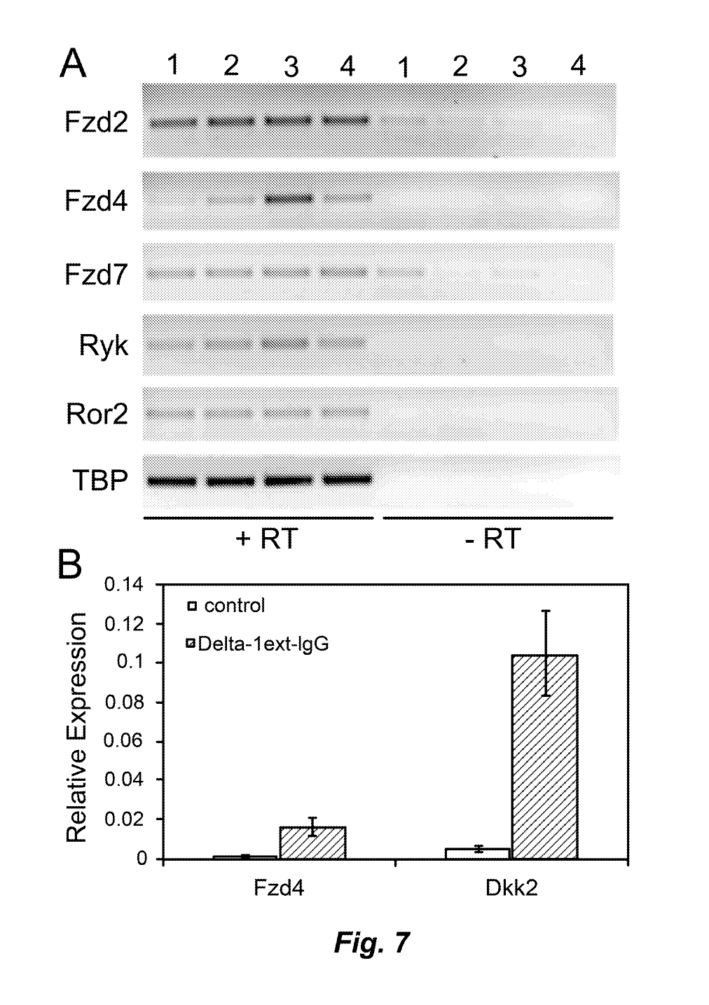

A survey by RT-qPCR of Wnt receptor expression in proliferating myoblasts and in myogenic precursor cells expanded on either Delta-1^{ext}-IgG or human IgG (as described in Example 1) demonstrated that Fzd2, Fzd4, Fzd7, Ror2, and Ryk were expressed in canine muscle derived cells (FIG. 7A). Activation of Notch signaling in the same cells increased expression of Fzd4, a mediator of non-canonical Wnt signaling, and Dkk2, an extracellular antagonist of canonical Wnt signaling (FIG. 7B). Wnt3a has been shown to stimulate proliferation of Pax7+ cells in vitro, yet Brack and colleagues demonstrated that treating muscle after injury with Wnt3a activated canonical Wnt signaling, and stimulated differentiation at the expense of myogenic progenitor proliferation (Brack et al., 2007 Science 317:807; Brack et al., 2008 Cell Stem Cell 2:50; see also Otto et al., 2008 J. Cell Sci. 121:2939). On the other hand, Wnt7a, acting through Fzd7 and the non-canonical pathway, enhanced proliferation and specifically expanded the murine satellite stem cell population (Pax7+Myf5-MyoD-) (LeGrand et al., 2009 Cell Stem Cell 4:535). Therefore, it is believed according to non-limiting theory that activating the canonical or non-canonical Wnt signaling pathway in cells expanded on Delta-1 ext-IgG will further expand cells early in myogenic lineage progression. Accordingly, certain embodiments contemplated by the present disclosure include the use of Wnt ligands and/or Wnt receptor agonists, in addition to Notch signaling, for expansion of muscle derived cells for transplant, such as the herein described myogenic precursor cells in which engraftment potential is preserved.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An ex vivo method for expanding myogenic precursor cells while preserving engraftment potential in one or more of said myogenic precursor cells, the method comprising activating Notch signaling in one or a plurality of myogenic precursor cells that are present in a population of cells isolated from skeletal muscle, said step of activating taking place in vitro under conditions and for a time sufficient for expansion of the myogenic precursor cells in the population of cells to obtain one or a plurality of myogenic precursor cells in which Notch signaling is detectably activated in a statistically significant manner to a greater degree than in control cells that do not undergo said step of activating, and thereby expanding the myogenic precursor cells while preserving engraftment potential in one or more of said cells.

2. The method of claim 1 wherein the step of activating Notch signaling comprises contacting the population of cells with an immobilized Notch ligand.

3. The method of claim 2 wherein the Notch ligand comprises a polypeptide selected from a eukaryotic Notch ligand delta family member and a eukaryotic Notch ligand serrate family member.

4. The method of claim 3 wherein the eukaryotic Notch ligand delta family member is selected from human delta-like-1 (DLL1, UniProt ID O00548 (SEQ ID NO: 1), Genbank ACH57449 (SEQ ID NO: 2), Genbank NP_{—}005609.3 (SEQ ID NO: 3)), delta-like-3 (DLL3, cDNA (var. 1) NM_{—}016941 (SEQ ID NO: 4); protein (var. 1) NP_{—}058637.1 (SEQ ID NO: 5); cDNA (var. 2) NM_{—}203486 (SEQ ID NO: 6); protein (var. 2) NP_{—}982353.1 (SEQ ID NO: 7)), delta-like-4 (DLL4, cDNA NM_{—}019074 (SEQ ID NO: 8); protein NP_{—}061947.1 (SEQ ID NO: 9)), Dlk1 (NP_{—}003827.3) (SEQ ID NO: 10), Dlk2 (NP_{—}076421.2 (SEQ ID NO: 12) (var. 1), NP_{—}996262.1 (SEQ ID NO: 13) (var. 2)), MAGP1/MFAP2 (NP_{—}059453.1 (SEQ ID NO: 16) (var. 1), NP_{—}002394.1 (SEQ ID NO: 17) (var. 2), NP_{—}001128719.1 (SEQ ID NO: 18) (var. 3), NP_{—}001128720.1 (SEQ ID NO: 19) (var. 4)), MAGP2/MFAP5 (NP_{—}003471.1) (SEQ ID NO: 24), JAG1 (NM_{—}000214 (SEQ ID NO: 26); protein NP_{—}000205.1 (SEQ ID NO: 27)) and JAG2 (NM_{—}002226 (SEQ ID NO: 28); protein NP_{—}002217.3 (SEQ ID NO: 29)).

5. The method of claim 2 wherein the Notch ligand comprises an extracellular domain of human delta-like-1 (DLL1, UniProt ID O00548 (SEQ ID NO: 1), Genbank ACH57449 (SEQ ID NO: 2), Genbank NP_{—}005609.3 (SEQ ID NO: 3)) or a polypeptide that has at least 80% sequence identity to said extracellular domain and is capable of activating Notch signaling.

6. The method of claim 2 wherein the immobilized Notch ligand comprises a fusion protein which comprises a Notch ligand polypeptide fused to a fusion domain polypeptide.

7. The method of claim 6 wherein the fusion domain polypeptide is selected from an immunoglobulin constant region polypeptide, a GST polypeptide, a streptavidin polypeptide, a maltose binding protein polypeptide, a c-myc polypeptide, a yeast Aga2p polypeptide, a filamentous phage coat protein polypeptide, a FLAG polypeptide, and a calmodulin binding peptide (CBP).

8. The method of claim 2 wherein the immobilized Notch ligand is expressed on cell surfaces of a feeder cell layer that is present during said step of contacting.

9. The method of claim 1 wherein detectably activated Notch signaling comprises a statistically significant increase in expression by the myogenic precursor cells of at least one marker gene selected from the group consisting of Hey1 (NM_{—}001002953 (SEQ ID NO: 30) (canine cDNA); NP_{—}001002953.1 (SEQ ID NO: 31) (canine protein); NM_{—}012258 (SEQ ID NO: 32) (human var. 1 cDNA); NP_{—}036390.3 (SEQ ID NO: 33) (human var. 1 protein); NM_{—}001040708 (SEQ ID NO: 34) (human var. 2 cDNA); NP_{—}001035798.1 (SEQ ID NO: 35) (human var. 2 protein), HeyL (NM_{—}014571 (SEQ ID NO: 36) (human cDNA); NP_{—}055386.1 (SEQ ID NO: 37) (human protein)) and Dtx4 (NM_{—}015177 (SEQ ID NO: 38) (human cDNA); NP_{—}055992.1 (SEQ ID NO: 39) (human protein)), relative to expression of the marker gene by myogenic precursor cells that do not undergo the step of activating Notch signaling.

10. The method of claim 1 wherein detectably activated Notch signaling comprises inhibition of differentiation of the myogenic precursor cells that manifests as one or more of (i) a statistically significant increase in expression by the myogenic precursor cells of at least one marker gene selected from the group consisting of Pax7 (NM_{—}002584 (SEQ ID NO: 40) (human cDNA); NP_{—}002575.1 (SEQ ID NO: 41) (human protein)), musculin (NM_{—}005098 (SEQ ID NO: 42) (human cDNA); NP_{—}005089.2 (SEQ ID NO: 43) (human protein)), Myf5 (NM_{—}005593 (SEQ ID NO: 44) (human cDNA); NP_{—}005584.2 (SEQ ID NO: 45) (human protein)), CXCR4 (NM_{—}001008540 (SEQ ID NO: 46) (human cDNA); NP_{—}001008540.1 (SEQ ID NO: 47) (human protein)) and syndecan4 (NM_{—}002999 (SEQ ID NO: 48) (human cDNA); NP_{—}002990.2 (SEQ ID NO: 49) (human protein)), relative to expression of the marker gene by myogenic precursor cells that do not undergo the step of activating Notch signaling, and (ii) a statistically significant decrease in expression by the myogenic precursor cells of at least one marker gene selected from the group consisting of myogenin (NM_{—}002479 (SEQ ID NO: 50) (human cDNA); NP_{—}002470.2 (SEQ ID NO: 51) (human protein)) and MyoD (NM_{—}002478 (SEQ ID NO: 52) (human cDNA); NP_{—}002469.2 (SEQ ID NO: 53) (human protein)), relative to expression of the marker gene by myogenic precursor cells that do not undergo the step of activating Notch signaling.

11. The method of any one of claims 1-10 which further comprises contacting a Wnt ligand, or a Wnt ligand receptor agonist, with the one or plurality of myogenic precursor cells in which Notch signaling is activated.

12. The method of claim 11 in which at least one of:
(a) the Wnt ligand is Dkk2;
(b) the Wnt ligand receptor agonist is capable of signaling via Fzd4;
(c) the Wnt ligand is selected from human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, Dkk-1, Dkk-2, Dkk-4, sFRP-1, sFRP-2, sFRP-3, sFRP4, sFRP-5, WIF-1, Norrin, R-spondin, and DkkL1; and
(d) the Wnt ligand receptor agonist is capable of activating a canonical or non-canonical Wnt signaling pathway via at least one of FZD1, FZD2, FZD3, FZD4, FZD5, FZD6, FZD7, FZD8, FZD9, LRP5, LRP6, ROR1, ROR2, RYK, MuSK, and a glypican.

13. An ex vivo method for expanding myogenic precursor cells while preserving engraftment potential in one or more of said myogenic precursor cells, the method comprising activating Notch signaling in one or a plurality of myogenic precursor cells that are present in a population of cells isolated from skeletal muscle by contacting the population of cells with an immobilized Notch ligand, said step of activating taking place in vitro under conditions and for a time sufficient for expansion of the myogenic precursor cells in the population to obtain one or more myogenic precursor cells in which Notch signaling is detectably activated in a statistically significant manner to a greater degree than in control cells that do not undergo said step of activating, and thereby expanding the myogenic precursor cells while preserving engraftment potential in one or more of said cells.

14. The method of claim 13 wherein the immobilized Notch ligand comprises a fusion protein which comprises (i) an extracellular domain of human delta-like-1 (DLL1, UniProt ID O00548 (SEQ ID NO: 1), Genbank ACH57449 (SEQ ID NO: 2), Genbank NP_{—}005609.3 (SEQ ID NO: 3)) or a polypeptide that has at least 80% sequence identity to said extracellular domain and is capable of activating Notch signaling, fused to (ii) an immunoglobulin constant region polypeptide.

15. The method of either claim 13 or claim 14 which further comprises contacting a Wnt ligand, or a Wnt ligand receptor agonist, with the one or plurality of myogenic precursor cells in which Notch signaling is activated.

16. The method of claim 15 in which at least one of:
(a) the Wnt ligand is Dkk2;
(b) the Wnt ligand receptor agonist is capable of signaling via Fzd4;
(c) the Wnt ligand is selected from human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, Dkk-1, Dkk-2, Dkk-4, sFRP-1, sFRP-2, sFRP-3, sFRP4, sFRP-5, WIF-1, Norrin, R-spondin, and DkkL1; and
(d) the Wnt ligand receptor agonist is capable of activating a canonical or non-canonical Wnt signaling pathway via at least one of FZD1, FZD2, FZD3, FZD4, FZD5, FZD6, FZD7, FZD8, FZD9, LRP5, LRP6, ROR1, ROR2, RYK, MuSK, and a glypican.

17. A composition comprising ex vivo expanded myogenic precursor cells in which engraftment potential is preserved, said composition being formed by a method which comprises activating Notch signaling in one or a plurality of myogenic precursor cells that are present in a population of cells isolated from skeletal muscle by contacting the population of cells with an immobilized Notch ligand, said step of activating taking place in vitro under conditions and for a time sufficient for expansion of the myogenic precursor cells in the population of cells to obtain one or a plurality of myogenic precursor cells in which Notch signaling is detectably activated in a statistically significant manner to a greater degree than in control cells that do not undergo said step of activating, and thereby expanding the myogenic precursor cells while preserving engraftment potential in one or more of said cells.

18. The composition of claim 17 that is formed by a method which further comprises contacting a Wnt ligand, or a Wnt ligand receptor agonist, with the one or plurality of myogenic precursor cells in which Notch signaling is activated.

19. The composition of claim 18 wherein in the method at least one of:
(a) the Wnt ligand is Dkk2;
(b) the Wnt ligand receptor agonist is capable of signaling via Fzd4;
(c) the Wnt ligand is selected from human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, Dkk-1, Dkk-2, Dkk-4, sFRP-1, sFRP-2, sFRP-3, sFRP4, sFRP-5, WIF-1, Norrin, R-spondin, and DkkL1; and
(d) the Wnt ligand receptor agonist is capable of activating a canonical or non-canonical Wnt signaling pathway via at least one of FZD1, FZD2, FZD3, FZD4, FZD5, FZD6, FZD7, FZD8, FZD9, LRP5, LRP6, ROR1, ROR2, RYK, MuSK, and a glypican.

20. A method for promoting muscle tissue regeneration in a mammal, comprising:
(a) activating Notch signaling, in one or a plurality of myogenic precursor cells that are present in a population of cells isolated from skeletal muscle, by contacting the population of cells with an immobilized Notch ligand, said step of activating taking place in vitro under conditions and for a time sufficient for expansion of the myogenic precursor cells in the population of cells to obtain one or a plurality of myogenic precursor cells in which Notch signaling is detectably activated, in a statistically significant manner to a greater degree than in control cells that do not undergo said step of activating, and thereby obtaining myogenic precursor cells having increased engraftment potential in a statistically significant manner relative to control cells that do not undergo said step of activating; and
(b) administering said myogenic precursor cells that have increased engraftment potential to a transplantation site in a mammal, and thereby promoting muscle regeneration.

21. The method of claim 20 wherein the immobilized Notch ligand comprises a fusion protein which comprises (i) an extracellular domain of human delta-like-1 (DLL1, UniProt ID O00548 (SEQ ID NO: 1), Genbank ACH57449 (SEQ ID NO: 2), Genbank NP_{—}005609.3 (SEQ ID NO: 3)) or a polypeptide that has at least 80% sequence identity to said extracellular domain and is capable of activating Notch signaling, fused to (ii) an immunoglobulin constant region polypeptide.

22. The method of either claim 20 or claim 21 which further comprises contacting a Wnt ligand, or a Wnt ligand receptor agonist, with the one or plurality of myogenic precursor cells in which Notch signaling is activated.

23. The method of claim 22 in which at least one of:
(a) the Wnt ligand is Dkk2;
(b) the Wnt ligand receptor agonist is capable of signaling via Fzd4;
(c) the Wnt ligand is selected from human Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, Dkk-1, Dkk-2, Dkk-4, sFRP-1, sFRP-2, sFRP-3, sFRP4, sFRP-5, WIF-1, Norrin, R-spondin, and DkkL1; and
(d) the Wnt ligand receptor agonist is capable of activating a canonical or non-canonical Wnt signaling pathway via at least one of FZD1, FZD2, FZD3, FZD4, FZD5, FZD6, FZD7, FZD8, FZD9, LRP5, LRP6, ROR1, ROR2, RYK, MuSK, and a glypican.
